SEQUENCE LISTING (10 sequences)

SEQ ID NO: 1 - DNA, 60 bp, Artificial Sequence
  Synthetic oligonucleotide
agtaggtagcgcgtcagtctcataatctgaaggtcgtgagttcgatcctcacacggggca

SEQ ID NO: 2 - DNA, 60 bp, Artificial Sequence
  Synthetic oligonucleotide
aggcgctggattaaggctccagtctcttcggaggcgtgggttcgaatcccaccgctgcca

SEQ ID NO: 3 - DNA, 60 bp, Artificial Sequence
  Synthetic oligonucleotide
agtggttatcacgttcgcctaacacgcgaaaggtccccggttcgaaaccgggcggaaaca

SEQ ID NO: 4 - DNA, 60 bp, Artificial Sequence
  Synthetic oligonucleotide
ggcgatggactagaaatccattggggtttccccgcgcaggttcgaatcctgccgactacg

SEQ ID NO: 5 - DNA, 20 bp, Artificial Sequence
  Synthetic oligonucleotide
tacccttgtgcctcgctcag

SEQ ID NO: 6 - DNA, 20 bp, Artificial Sequence
  Synthetic oligonucleotide
gagaagatcagccggcgttt

SEQ ID NO: 7 - DNA, 21 bp, Artificial Sequence
  Synthetic oligonucleotide
catgtacgttgctatccaggc

SEQ ID NO: 8 - DNA, 21 bp, Artificial Sequence
  Synthetic oligonucleotide
ctccttaatgtcacgcacgat

SEQ ID NO: 9 - DNA, 20 bp, Artificial Sequence
  Synthetic oligonucleotide
ggctgtattcccctccatcg

SEQ ID NO: 10 - DNA, 22 bp, Artificial Sequence
  Synthetic oligonucleotide
ccagttggtaacaatgccatgt

(19) United States
(12) Patent Application Publication
(10) Pub. No.: US 2021/0369858 A1
(43) Pub. Date: Dec. 2, 2021

(54) USE OF EXOSOMES FOR TARGETED DELIVERY OF THERAPEUTIC AGENTS

(71) Applicant: BOARD OF REGENTS, THE UNIVERSITY OF TEXAS SYSTEM, Austin, TX (US)

(72) Inventors: Raghu KALLURI, Houston, TX (US); Valerie LEBLEU, Houston, TX (US); Pedro CORREA DE SAMPAIO, Houston, TX (US)

(21) Appl. No.: 17/042,007

(22) PCT Filed: Mar. 28, 2019
(86) PCT No.: PCT/US2019/024603

§ 371 Date: Sep. 25, 2020

Related U.S. Application Data
(60) Provisional application No. 62/546,057, filed on Aug. 16, 2017.

(51) Int. Cl. A61K 47/69; A61K 9/127; A61K 47/64; A61K 31/7105; A61K 39/395; A61P 35/00

(52) U.S. Cl. CPC A61K 47/6911; A61K 9/127; A61K 47/6425; A61K 48/00; A61K 39/39558; A61P 35/00; A61K 31/7105

(57) ABSTRACT

Provided herein are methods of using exosomes that function like minicells to deliver therapeutic agents to diseased or disordered cells. In particular, the exosomes can be targeted to particular areas of the body using growth factor gradients. These gradients also serve to trigger expression of proteins inside the exosomes, from transfected nucleic acids, at the desired target.

### REFERENCE TO RELATED APPLICATIONS

The present application claims the priority benefit of U.S. provisional application No. 62/649,057, filed Mar. 28, 2018, the entire contents of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Mar. 21, 2019, is named UTFC.P1363WO_ST25.txt and is 3 kilobytes in size.

### BACKGROUND

### 1. Field

The present invention relates generally to the fields of biology, medicine, and oncology. More particularly, it concerns the use of exosomes to target delivery of therapeutic agents to diseased or disordered cells.

### 2. Description of Related Art

Exosomes are small extracellular vesicles (EVs) with a lipid bilayer that contain proteins and polynucleotides, including messenger RNAs (mRNAs), non-coding RNAs and double-stranded genomic DNA (Kalluri, 2016; Raposo and Stoorvogel, 2013). After their initial discovery as byproducts of reticulocyte differentiation (Harding et al., 1984; Raposo and Stoorvogel, 2013), it is now generally accepted that exosomes are secreted by virtually all mammalian cells and found in all body fluids (El-Andaloussi et al., 2013; Kalluri, 2016).

Exosomes are part of a larger group of extracellular vesicles, which also include microvesicles and apoptotic bodies (Colombo et al., 2014). Amongst extracellular vesicles, exosomes are typically distinguished through their unique biogenesis via the endocytic pathway. Endocytic vesicles mature into late endosomes, also known as multivesicular bodies, which contain a number of intracellular vesicles (ILVs) generated through invagination of the endosomal membrane. Through a likely fusion of these multivesicular bodies with the plasma membrane, exosomes are released into the extracellular space and enter circulation (Bastos et al., 2017; Colombo et al., 2014). As a result of their endocytic origin, exosomes membranes have a similar polarity to cellular membranes, containing membrane proteins anchored with their intracellular domains facing the lumen and the extracellular domains facing the extracellular space. While the protein content of exosomes varies depending on their cellular origin, several proteins seem to be generally enriched. These include members of the tetraspanin family and components of the endocytic and ILV maturation pathways, such as Rab proteins and members of the ESCRT complex. Interestingly, different proteomics studies performed with exosomes derived from many different cell types have identified many constituents associated with the protein translation machinery, such as eukaryotic initiation factors, ADP ribosylation factors, ribosomal proteins (Pisitkun et al., 2004; Valadi et al., 2007). Additionally, a subset of transcriptional and translation regulators identified in exosomes by proteomic analysis has been suggested to be delivered to recipient cells, altering their pattern of gene and protein expression (Ung et al., 2014).

Amongst the proteins commonly identified in exosomes are growth factor receptors, such as the epithelial growth factor receptor (EGFR). EGFR is a member of the ErbB family of growth factor receptors, which also includes HER2, HER3 and HER4 (Seshacharyulu et al., 2012). Upon binding one of its ligands, such as the epitheial growth factor (EGF), the receptor dimerizes, forming either homodimers or heterodimers with other members of the ErbB family (Seshacharyulu et al., 2012). This dimerization activates the receptor's intrinsic kinase activity, leading to the autophosphorylation of different key tyrosine residues on its cytoplasmic domain. This authophosphorylation reaction recruits different adaptor proteins containing SH2 and PTB (phosphotyrosine binding) domains, such as Shc and GRB2, which mediate different downstream signaling activities, including the synthesis of relevant proteins (Normanno et al., 2006; Tomas et al., 2014). Phosphorylated EGFR is ultimately ubiquitinated and transported to the endosomal pathway, from which it will either recycle back to the membrane or remain in the late endosomal pathway leading to integration into multivesicular bodies or lysosomal degradation (Tomas et al., 2014). Since multivesicular bodies originate exosomes, it is likely that the post-phosphorylation recycling of EGFR (and other growth factors) contribute to their membrane localization in these extracellular vesicles.

EGFR signaling has been shown to be important for the progression of different malignancies, such as glioblastoma, lung cancer, and breast cancer (Lim et al., 2016; Liu et al., 2012; Masuda et al., 2012; Morgillo et al., 2016; Westphal et al., 2017; Zhang et al., 2013). Perhaps for this reason, most studies of EGFR in exosomes have been performed in the context of cancer development. EGFR signaling has particularly been implicated in the patterns of cellular uptake and secretion of exosomes from different origins. In mantle cell carcinoma cells, incubation with gefitinib (an EGFR inhibitor) has been shown to dramatically decrease the rate of exosomes uptake (Hazan-Halevy et al., 2015). Treatment of lung cancer cells with gefitinib leads to an increased secretion of exosomes, which mediate horizontal transfer of cisplatin resistance (Li et al., 2016). The transfer of EGFR via cancer cell-derived exosomes has also been known to cause alterations in components of the microenvironment, such as endothelial cells and T cells (Al-Nedawi et al., 2009; Huang et al., 2013). More recently, exosomes derived from gastric cancer cells containing EGFR were shown to be delivered to stromal cells in the liver, mediating metastasis (Zhang et al., 2017). Finally, exosomes derived from breast cancer cells were shown to contain functional phosphorylated forms of EGFR, which can be transferred to monocytes mediating their survival through activation of the ERK pathway (Song et al., 2016).

While the delivery of EGFR and members of the protein translation machinery by exosomes seems to have clear biological importance in the context of cell-cell communications, these properties may be harnessed to target the delivery of therapeutic agents to certain tissues and to induce therapeutic protein production at the desired delivery site.

### SUMMARY

Here, protein synthesis was induced in exosomes through growth factor stimulation. Exosomes that contain DNA, RNA, and proteins, can respond to biological stimuli, and initiate properties such as migration, multiplication, initiation of signaling network/cascade, transcription, and protein translation. Thus, in one embodiment, provided herein are exosomes with the ability to function like minicells. As discussed further below, these minicell-like exosomes can be employed in numerous therapeutic means to treat various disease and/or disorders.

In one embodiment, provided herein are methods of treating a disease or disorder in a patient in need thereof, the method comprising (a) obtaining exosomes having a growth factor receptor on their surface; (b) transfecting the exosomes with a nucleic acid encoding a therapeutic protein; (c) administering the transfected exosomes to a patient; (d) providing a growth factor gradient at a site of the disease or disorder to attract the exosomes to the site and stimulate production of the therapeutic protein at the site, thereby treating the disease in the patient.

In some aspects, the method is further defined as a method of administering a therapeutic protein to a diseased cell in a patient. In some aspects, the exosomes obtained in step (a) are obtained from a body fluid sample obtained from the patient. In some aspects, the body fluid sample is blood, lymph, saliva, urine, cerebrospinal fluid, bone marrow aspirates, eye exudate/tears, or serum. In some aspects, the nucleic acid is an mRNA, a plasmid, or a cDNA.

In some aspects, the disease or disorder is cancer, an injury, an autoimmune disorder, a neurological disorder, a gastrointestinal disorder, an infectious disease, a kidney disease, a cardiovascular disorder, an ophthalmic disorder, a skin disease or disorder, a urogenital disorder, or a bone disease or disorder. In certain aspects, the cancer is a breast cancer, lung cancer, head & neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer or skin cancer. In some aspects, the site of the disease or disorder is a tumor. In some aspects, the cancer is metastatic. In certain aspects, the site of the disease or disorder is a metastatic node.

In some aspects, the therapeutic protein is a kinase, a phosphatase, or a transcription factor. In certain aspects, the therapeutic protein corresponds to a wildtype version of a protein that is mutated or inactivated in a cell at the site of the disease or disorder. In certain aspects, the therapeutic protein corresponds to a dominant negative version of a protein that is hyperactive in a cell at the site of the disease or disorder. In certain aspects, the disease or disorder is cancer, wherein the therapeutic protein is a tumor suppressor. In some aspects, the exosomes comprise CD47 on their surface. In some aspects, transfection comprises electroporation.

In some aspects, the method further comprises administering at least a second therapy to the patient. In some aspects, the second therapy comprises a surgical therapy, chemotherapy, radiation therapy, cryotherapy, hormonal therapy, or immunotherapy.

In one embodiment, methods are provided of treating a disease or disorder in a patient in need thereof, the method comprising (a) obtaining exosomes having a growth factor receptor on their surface; (b) transfecting the exosomes with therapeutic agent; (c) administering the transfected exosomes to a patient; (d) providing a growth factor gradient at a site of the disease or disorder to attract the exosomes to the site and deliver the therapeutic agent to the site, thereby treating the disease in the patient.

In some aspects, the method is further defined as a method of administering a therapeutic agent to a diseased cell in a patient. In some aspects, the exosomes obtained in step (a) are obtained from a body fluid sample obtained from the patient. In certain aspects, the body fluid sample is blood, lymph, saliva, urine, cerebrospinal fluid, bone marrow aspirates, eye exudate/tears, or serum.

In some aspects, the therapeutic agent is a therapeutic protein, an antibody, an inhibitory RNA, a gene editing system, or a small molecule drug. In certain aspects, the antibody binds an intracellular antigen. In certain aspects, the antibody is a full-length antibody, an scFv, a Fab fragment, a (Fab)2, a diabody, a triabody, or a minibody. In certain aspects, the inhibitory RNA is a siRNA, shRNA, miRNA, or pre-miRNA. In certain aspects, the gene editing system is a CRISPR/Cas system. In certain aspects, the therapeutic protein is a kinase, a phosphatase, or a transcription factor. In certain aspects, the therapeutic protein corresponds to a wildtype version of a protein that is mutated or inactivated in a cell at the site of the disease or disorder. In certain aspects, the therapeutic protein corresponds to a dominant negative version of a protein that is hyperactive in a cell at the site of the disease or disorder. In some aspects, the small molecule drug is an imaging agent.

In some aspects, the disease or disorder is cancer, an injury, an autoimmune disorder, a neurological disorder, a gastrointestinal disorder, an infectious disease, a kidney disease, a cardiovascular disorder, an ophthalmic disorder, a skin disease or disorder, a urogenital disorder, or a bone disease or disorder. In certain aspects, the cancer is a breast cancer, lung cancer, head & neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer or skin cancer. In some aspects, the site of the disease or disorder is a tumor. In some aspects, the cancer is metastatic. In some aspects, the site of the disease or disorder is a metastatic node. In some aspects, the disease or disorder is cancer, wherein the therapeutic protein is a tumor suppressor. In some aspects, the disease or disorder is cancer, wherein the therapeutic agent is an inhibitory RNA targeting an oncogene.

In some aspects, the exosomes comprise CD47 on their surface. In some aspects, transfection comprises electroporation. In some aspects, the method further comprises administering at least a second therapy to the patient. In some aspects, the second therapy comprises a surgical therapy, chemotherapy, radiation therapy, cryotherapy, hormonal therapy, or immunotherapy.

In further aspects, exosomes for use according to the embodiments are comprised in a tissue scaffold matrix. For example, such a matrix may be a synthetic matrix, such a matrix that degradable or can be absorbed in tissues. In further aspects, the matrix may be a living tissue matrix. In some aspects, a exosomes of the embodiments are cultured in a matrix.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-E. EGFR phosphorylation in exosomes. FIG. 1A—Immunoblot of EGFR expression on exosomes obtained from different human and murine cell lines. The exosomes marker CD81 is used as a loading control, and to confirm the exosomal origin of protein extracts. FIG. 1B—Immunoblot showing phosphorylation of EGFR on exosomes derived from MDA-MB-231 cells, but not MCF10A cells, after incubation with 500 ng/ml rhEGF for 15 minutes at 37° C. Phosphorylation levels are detected using an antibody specific for the Tyr1068 residue of EGFR. EGFR levels are shown as a loading control, to confirm differences in phosphorylation. Band densitometry quantification was performed using ImageJ software. FIG. 1C—Immunoblot showing the presence of EGFR adaptor proteins Shc and GRB2, as well increased levels of phosphorylated-ERK protein, in exosomes derived from MDA-MB-231 cells, with and without rhEGF stimulation for 15 minutes at 37° C. The exosomes marker CD81 is used as a loading control, and to confirm the exosomal origin of protein extracts. FIG. 1D—GRB immunecomplexes were obtained from protein extracts of MDA-MB-231 exosomes, with and without incubation with 500 ng/ml rhEGF for 15 minutes at 37° C., using a GRB2 specific antibody. Immunoblot analysis of the immunocomplexes shows association of GRB2 with EGFR only upon rhEGF stimulation. Non-specific isotype control IgG was used as a negative control for the GRB2 pulldown. Equal volumes of the stimulated and unstimulated extracts were probed for β-actin as input control. FIG. 1E—Similar experiment using an Shc antibody for the pull down experiment in duplicates, also showing association with EGFR only after stimulation with 500 ng/ml rhEGF for 15 minutes at 37° C. Non-specific isotype control IgG was used as a negative control for the Shc pulldown. Equal volumes of the stimulated and unstimulated extracts were probed for bactin as an input control.

FIGS. 2A-G. EGFR phosphorylation alters the content of exosomes. FIG. 2A—Luciferase-based ATP determination assay ran on protein extracts obtained from exosomes either unstimulated or after rhEGF stimulation for 15 minutes at 37° C. Luciferin-derived luminescence was measured in a plate reader and is presented as arbitrary units. Significance was determined with a Mann-Whitney test (n=4). FIG. 2B—Immunoblot analysis of exosomes from MDA-MB-231 cells incubated with 500 ng/ml rhEGF at 37° C. for 48 h, showing an increase in both pEGFR and GRB2 levels when compared to unstimulated exosomes. FIG. 2C—Cellular component association analysis of mass spectrometry data obtained for MDA-MB-231 exosomes unstimulated or after incubation with 500 ng/ml EGF at 37° C. for 48 h. A list of significant proteins identified for stimulated and unstimulated exosomes was obtained and used as input for the open access FunRich functional enrichment analysis tool in order to identify the subcellular origin of the identified proteins. FIG. 2D—Venn diagram depicting the overlap in proteins identified in control MDA-MB-231 exosomes and exosomes incubated with 500 ng/ml rhEGF at 37° C. for 48 h. FIG. 2E—Individual EGFR and GRB2 protein scores obtained from mass spectrometry analysis of MDA-MB-231 exosomes with or without incubation with 500 ng/ml rhEGF for 48 h. FIG. 2F—BCA analysis of protein extracts obtained from control MDA-MB-231 exosomes and exosomes incubated with 500 ng/ml rhEGF at 37° C. for 48 h. Significance was determined with a Mann-Whitney test (n=3). FIG. 2G—Immunoblot analysis of β-actin expression on protein extracts obtained from control MDA-MB-231 exosomes and exosomes incubated with 500 ng/ml and 1000 ng/ml rhEGF at 37° C. for 48 h. (*p<0.05, **p<0.01, ***p<0.005, ****p<0.0001).

FIGS. 3A-F. Exosomes contain functional components for transcription and translation. FIG. 3A—Ultra-performance liquid chromatography-mass spectrometry (UPLCMS) was used to detect free amino acids in MCF10A-, MDA-MB-231-, HDF, E10-, and NIH-3T3-derived exosomes. Data are represented as a HeatMap using normalized signal intensities (log 10), with hotter colors corresponding to higher intensity levels, as represented in the color legend. FIG. 3B—Immunoblot of eIF4A1, eIF3A, and eIF1A in protein extracts of exosomes obtained from E10-, NIH-3T3-, MCF10A-, HDF-, and MDA-MB-231-derived exosomes. CD9 was used as a loading control. FIG. 3C—In vitro translation assay using protein lysates from MCF10A- and MDA-MB-231-derived exosomes incubated with the pEMT7-GFP cDNA expression plasmid. Protein lysates obtained from cells were used as controls. FIG. 3D—Immunoblot analysis of RNA Polymerase II in exosomes protein extracts, with CD9 shown as a loading control. FIG. 3E—Autoradiography of exosomes derived from MDA-MB-231 and E10 cells cultured in the presence of ³⁵S-methionine. Exosomes only, as well as exosomes cultured in the presence of ³⁵S-methionine and cycloheximide, were used as controls. FIG. 3F—BCA quantification of protein extracts obtained from exosomes immediately after isolation, or after incubation in cell-free conditions for 24 h and 48 h. Significance was determined with a one-way ANOVA followed by Tukey's multiple comparisons test (*p<0.05, **p<0.01, ***p<0.005, ****p<0.0001, n=3).

FIGS. 4A-J. Exosomes synthesize new proteins through DNA transcription and cap-dependent mRNA translation. FIG. 4A—qPCR analysis of GFP mRNA levels in exosomes isolated from MDA-MB-231 cells and either non-electroporated, mock electroporated, or electroporated with a pCMV-GFP plasmid with or without the presence of α-amanitin. Expression levels were normalized to GAPDH. FIG. 4B—Transmission electron microscopy images of immunogold labeling, using anti-GFP antibody, of exosomes electroporated with GFP plasmid and incubated in cell-free conditions for 48 h (bottom row). Secondary antibody only was used as a negative control (top row). Gold particles are depicted as black dots. Scale bar, 100 nm. FIG. 4C—Immunoblot of GFP protein expression in exosomes electroporated with a pCMV-GFP plasmid and incubated for 12 hours, 2 days, or 1 week at 37° C. Exosomes only and mock-electroporated exosomes were used as negative controls. The exosomes marker TSG101 was used as a loading control for the presence of exosomes. FIG. 4D—Immunoblot of GFP protein expression in exosomes electroporated with GFP plasmid and incubated for several periods of time up to one month. Non-electroporated exosomes were used as negative controls. The exosomes marker CD63 was used as a loading control for the presence of exosomes. FIG. 4E—Immunoblot of GFP protein expression in exosomes electroporated with a GFP plasmid immediately after isolation (0 h) or after incubation in cell-free conditions (24 h) and cultured as previously described. Mock electroporated exosomes were used as negative controls. The exosomes marker TSG101 was used as a loading control for the presence of exosomes. FIG. 4F—Immunoblot of GFP protein expression in exosomes electroporated with a pCMV-GFP plasmid and cultured with the translation inhibitor cycloheximide. Exosomes only and exosomes mock electroporated were used as negative controls. TSG101 was used as a loading control for the presence of exosomes. Band densitometry was performed using ImageJ software. FIG. 4G—Immunoblot of GFP protein expression in exosomes electroporated with a GFP plasmid and cultured with the transcription inhibitor α-amanitin. Exosomes only and exosomes mock electroporated were used as negative controls. TSG101 was used as a loading control for the presence of exosomes. Band densitometry was performed using ImageJ software. FIG. 4H—Schematic representation of the bicistronic plasmid used as a reporter for cap-dependent or cap-independent translation (pCDNA3-rLuc-polIRESfLuc). FIG. 4I—Activities of renilla (r-Luc) and firefly luciferase (f-Luc) measured by bioluminescence after 48 h incubation of exosomes electroporated with the bicistronic plasmid. Non-electroporated exosomes were used as negative controls. FIG. 4J—Luminescence counts measured from exosomes incubated for 48 h after electroporation with or without a plasmid with firefly luciferase expressed under a CMV promoter.

FIGS. 5A-E. Protein translation in exosomes generates functional proteins and can be increased by growth factor stimulation. FIG. 5A—Confocal microscopy showing the presence of GFP in MCF10A electroporated cells as well as in MCF10A cells, previously treated with cycloheximide, incubated with MDA-MB-231-derived exosomes electroporated with a pCMV-GFP plasmid and pre-incubated for 48 h. MCF10A cells treated with non-electroporated MDA-MB-231-derived exosomes were used as a negative control. FIG. 5B—Immunoblot analysis of GFP expression on protein lysates from MDA-MB-231-derived exosomes electroporated with a p53-GFP expression plasmid. Non-electroporated exosomes were used as negative controls. TSG101 was used as a loading control for the presence of exosomes. FIG. 5C—p21 mRNA expression in MDA-MB-231 cells treated with mock electroporated MDA-MB-231-derived exosomes or exosomes electroporated with the p53-GFP plasmid with and without the presence of cycloheximide. Expression levels were normalized to the housekeeping gene GAPDH. FIG. 5D—Immunoblot of exosomes isolated from MDA-MB-231 cells, incubated with 100 μg/ml of the translation inhibitor cycloheximide. Exosomes lysates were probed for β-actin and GAPDH. Band densitometry quantification was performed using ImageJ software. FIG. 5E—Immunoblot of GFP protein expression in exosomes electroporated with a pCMV-GFP plasmid, and then incubated in the presence of different concentrations of rhEGF at 37° C. for 48 h. Exosomes mock electroporated and exosomes without rhEGF incubation are shown as negative controls. The exosomes marker CD81 is used as a loading control. Band densitometry quantification was performed using ImageJ software.

FIG. 6A-C. Exosomes derived from MDA-MB-231 cells demonstrate chemotaxis towards a gradient of growth factors. FIG. 6A—Schematic demonstrating the set up for exosomes retrograde migration assay. In short, 10×10⁹ exosomes isolated from MDA-MB-231 cells were placed in the bottom well of a Corning Transwell® system. An HTS Transwell® insert with 400 nm pores was placed on top of the exosomes suspension containing either PBS, 20% FBS, or 10,000 ng/ml rhEGF and incubated at 37° C. The number of exosomes on the top insert was measured by Nanosight NTA after different time points to assess exosomes motility. FIGS. 6B&6C—Quantification of MDA-MB-231 exosomes on the top insert of the retrograde migration assay, after 4 h (FIG. 6B) and 24 h (FIG. 6C) incubation at 37° C., by Nanosight NTA. Significance was determined with a one-way ANOVA followed by Newman-Keuls multiple comparison test. (*p<0.05, **p<0.01, ***p<0.005, ****p<0.0001, n=3).

FIGS. 7A-D. Tumor-bearing mice show increased protein synthesis in delivered exosomes. FIG. 7A—Schematic depicting the experimental plan for the in vivo translation experiment. In short, female Balb/C mice were injected with 4T1 tumor orthotopically and the tumor was allowed to grow to 500 mm³, after which mice were injected with 30 billion MDA-MB-231 exosomes electroporated with a pCMV-mCherry plasmid. The mice were euthanized 12 h after exosomes injection and serum was collected for exosomes extraction. FIG. 7B—Graphic showing the tumor growth of mice injected with 4T1 tumors and electroporated exosomes, or 4T1 tumors alone, showing comparable growth kinetics. FIG. 7C—Nanosight NTA analysis of exosomes extracted from the serum of healthy mice injected with electroporated exosomes, as well as 4T1 tumor-bearing mice injected with electroporated exosomes and 4T1 tumor bearing mice with no exosomes injection. All exosomes show similar size peaks, around 100 nm. FIG. 7D—Nanosight NTA quantification of serum exosomes shown in FIG. 7C, showing no significant differences in the exosomes amount obtained from the serum of different animals, but a trend towards more exosomes in 4T1 tumor-bearing mice injected with MDA-MB-231 electroporated exosomes.

FIGS. 8A-G. Exosomes characterization. FIG. 8A—Nanoparticle tracking analysis of exosomes collected from MDA-MB-231 cells, obtained using the Nanosight NTA 2.1 Analytical Software. Left graph represents the size distribution of particles in solution showing a mean size of 104 nm and also showing no peaks at larger sizes. Right graph represents the distribution by size and concentration of particles in solution. FIG. 8B—Atomic Force Microscopy image of exosomes (left image). Right graph represents the distribution of particles in the area analyzed. FIG. 8C—Transmission electron micrograph of MDA-MB-231 exosomes. Scale bar—100 nm. FIG. 8D—Transmission electron micrograph of immunogold labeled MDA-MB-231 exosomes using anti-CD9 antibody. Gold particles are depicted as black dots. Scale bar—100 nm. FIG. 8E—Immunoblot analysis of exosomes markers CD9, CD63, and TSG101 in protein extracts of exosomes obtained from different cell lines. FIG. 8F—Imaging Flow Cytometry analysis of exosomes from MDA-MB-231 cells coupled to 0.4 μm beads, using antibodies for markers CD9, CD81, CD82, and CD63. FIG. 8G—Representative images of LB culture plates incubated with E. coli and either MDA-MB-231 (top) or MCF10A (bottom) exosomes, showing colony formation on the E. coli inoculated sides (left) and not on the exosomes inoculated sides (right).

FIGS. 9A-B. EGFR phosphorylation and downstream biological activity in exosomes from MDA-MB-231 cells. FIG. 9A—Immunoblot of protein extracts obtained from MDA-MB-231 cells and probed for p-EGFR and GRB2. β-actin is used as a loading control. FIG. 9B—Immunoblot of immunocomplexes obtained with an anti-EGFR antibody pull down of protein lysates from MDA-MB-231 exosomes with or without incubation with 500 ng/ml at 37° C. for 15 minutes. Immuncomplexes were probed for GRB2. Non-specific isotype control IgG was used as a negative control for the GRB2 pulldown. Equal volumes of the stimulated and unstimulated extracts were probed for β-actin as input control.

FIG. 10. Proteomics analysis of exosomes derived from various cells. Heatmap representing the binary identification of all individual proteins included in the Protein Translation pathway in the Reactome (Croft et al., 2014) database, in mass spectrometry data obtained from mouse liver cells (Valadi et al., 2007), mouse fibroblasts (Luga et al., 2012), human colorectal cancer cells (Choi et al., 2012), human plasma (Kalra et al., 2013), human thymic tissue (Skogberg et al., 2013), and human urine (Gonzales et al., 2009). Black represents the presence and white represents the absence of each protein in each dataset. The summary column represents how ubiquitous each protein is in all analyzed datasets, with warmer colors representing a more widespread distribution among different types of exosomes.

FIGS. 11A-B. Proteomics analysis of exosomes from various origins. FIG. 11A—Heatmap representing the number of proteins identified in mass spectrometry data obtained from mouse liver cells (Valadi et al., 2007), mouse fibroblasts (Luga et al., 2012), human colorectal cancer cells (Choi et al., 2012), human plasma (Kalra et al., 2013), human thymic tissue (Skogberg et al., 2013), and human urine (Gonzales et al., 2009) that associate with different pathways related to protein translation in the Reactome (Croft et al., 2014) database. Warmer colors represent higher numbers of proteins identified per pathway. FIG. 11B—Heatmap representing the protein score of proteins associated with protein translation identified in mass spectrometry obtained from exosomes isolated from HDF, NIH 3T3, MDA-MB231, MCF10A, and E10 cells. Warmer colors represent a higher protein score.

FIGS. 12A-C. Exosomes contain nucleic acids and proteins associated with the protein translation machinery. FIG. 12A—RNA extracted from exosomes of NIH-3T3, E10, 67NR, 4T1, HDF, MCF10A, MCF7, and MDA-MB-231 cell lines were used to quantify 18S and 28S rRNAs by qPCR. Expression levels of the rRNAs were normalized to U6 snRNA expression. The bars in each group represent, from left to right, NIH 3T3, E10, 67NR, 4T1, HDF, MCF10A, MCF7, and MDA-MB-231. FIG. 12B—RNA extracted from 4T1 exosomes and cells were used to identify the presence of tRNAMet, tRNAGly, tRNALeu, tRNASer, and tRNAVal by digital qPCR. The bars in each group represent, from left to right, Leu, Met, Val, Ser, and Gly. FIG. 12C—Immunoprecipitation of eIF4A1 showing presence of eIF3A MCF10A and MDA-MB-231-derived exosomes. MB231 and MCF10A cell lysates were used as positive controls. The exosomes marker CD82 was used as a loading control.

FIGS. 13A-E. DNA transcription and mRNA translation in exosomes derived from MCF10A and MDA-MB-231 cells. FIG. 13A—Immunoblot of GFP protein expression in exosomes isolated from MCF10A cells, electroporated with a pCMV-GFP plasmid and incubated at 37° C. for different periods of time. Exosomes only and mock electroporated exosomes were used as negative controls. CD63 was used as a loading control and to confirm the presence of exosomes. FIG. 13B—Plot depicting the amount of green exosomes detected by NanoSight in MCF10A-derived exosomes electroporated with a GFP plasmid. MCF10A-derived exosomes, MCF10A-derived mock electroporated exosomes as well as exosomes electroporated with α-amanitin and cycloheximide were used as negative controls. FIG. 13C—Flow cytometry analysis of beads attached to exosomes after electroporation with a GFP plasmid using increasing voltages, showing the percentages of beads with green fluorescent signal. FIG. 13D—Immunoblot of ovalbumin protein levels in MDA-MB-231 exosomes electroporated with a pCMV-Ova plasmid and incubated at 37° C. for 48 h. β-actin was used as a loading control. FIG. 13E—p21 mRNA expression in MDA-MB-231 cells treated with mock electroporated MDA-MB-231-derived exosomes or exosomes electroporated with the p53-GFP plasmid and either added to the cells immediately (0 h) or allowed to incubate in cell conditions at 37° C. for 48 h before treatment (48 h). Exosomes were added to the cells for either 30 minutes or 48 hours before RNA extraction. Expression levels were normalized to the housekeeping gene GAPDH.

### DETAILED DESCRIPTION

Extracellular vesicles (EVs), including exosomes, are nano-sized intercellular communication vehicles having a lipid bilayer that encloses cytosol-like material. Exosomes participate in several physiological processes and contain DNA, RNA, and proteins. It is generally assumed that all contents in exosomes are derived from cells and they remain as such in the exosomes until they enter other cells and deposit their contents. Exosomes are released by all cells in large numbers and are considered as garbage bags that carry cellular constituents into the extracellular space as a payload without any biological significance for exosomes themselves per se.

Provided here are exosomes that behave like minicells and exhibit the ability to biologically respond to stimuli and multiply and migrate just as cells do but without a defined nucleus. These exosomes exhibit chemotaxis towards serum factors and upon stimulation with growth factors such as EGF, will phosphorylate the EGFR receptor on their surface and initiate a signaling cascade that leads to transcription and translation of new proteins. When these exosomes are injected into tumor bearing mice, they preferentially accumulate in the tumors. Collectively, the capacity for protein translation and growth factor response by these exosomes provides them a functional role in tissue homeostasis and modulation of disease.

### I. ASPECTS OF THE PRESENT INVENTION

Extracellular vesicles, and in particular exosomes, have gained much attention over the last few years with identification of several constituents such as DNA, RNA, and proteins. Moreover, exosomes have been implicated in influencing many diverse biological processes via transfer of its content into recipient cells in different tissues, and facilitating a unique form of cell-cell communication (Bastos et al., 2017). Alternatively, the potential of exosomes as delivery vehicles for therapeutics, particularly in the context of cancer or neurological pathologies was also reported (El-Andaloussi et al., 2012; Kamerkar et al., 2017). However, the exact patterns of systemic distribution and organ tropism of exosomes are still not fully understood.

However, the nuclear and cytoplasmic components of the exosomes are not just used for passive transfer to recipient cells but can respond to external stimuli to phosphorylate growth factor receptors, such as EGFR, and generate new proteins via active transcription and translation. External stimulation of exosomes can initiate de novo biological activities such as retrograde migration. It is conceivable that exosomes may function like mini cells, albeit primitive with respect to their fine-tuned operations in response to external stimuli. In fact, recently it has been suggested that exosomes could potentially constitute extant representations of protocellular ribosomes, for which they would need to contain rRNAs, which is confirm in this study (Sinkovics, 2015). Exosomes are biologically responsive and migrate towards growth factor gradients in an active manner. Actin remodeling might be involved. The patterns of actin polymerization could therefore constitute an interesting target in the modulation of exosomes biodistribution.

Vesicles such as prostasomes obtained from different species have been shown to contain different components of the glycolytic pathway, which allow them to produce ATP in cell-free conditions (Ronquist et al., 2013a; Ronquist et al., 2013b). While direct transcription in exosomes has not been previously reported, a study showed that exosomes from bovine milk infected with bovine leukemia virus have been shown to exhibit reverse transcriptase activity (Yamada et al., 2013). It was also recently demonstrated that independent production of mature miRNAs in exosomes isolated from cancer cells is possible (Melo et al., 2014). Here, exosomes were demonstrated to possess an intrinsic capacity for de novo synthesis of functional proteins via DNA transcription coupled with mRNA translation. Platelets can translate proteins from mRNA molecules remaining within them after megakaryocyte differentiation (Weyrich et al., 2004). Nevertheless, DNA transcription resulting in new mRNA molecules is not reported in platelets. Additionally, foci of mRNA translation activity, associated with polyribosomes and mRNA binding proteins, is observed in dendritic spines even when severed from the major body of the cell (Aakalu et al., 2001; Smith et al., 2001; Steward and Levy, 1982). It is therefore clear that some cellular structures have preserved the capacity for protein biosynthesis in the absence of a nucleus, perhaps in order to support their specific biological functions in a rapid manner. Apart from protein translation, exosomes are capable of DNA transcription via RNA polymerase II. It is well established that basic transcription of naked DNA in nucleosome-free regions is possible with minimal components of transcriptional machinery, namely only RNA Pol II and a cocktail of six general transcription factors (GTFs) (Lorch et al., 2014; Nagai et al., 2017). It has also been shown that exosomes contain a plethora of transcription factors that can be delivered to cells in order to alter their patterns of protein expression (Ung et al., 2014). It is therefore conceivable that exosomes contain naked DNA residues unbound by chromatin, which could undergo transcription in the presence of these minimal transcriptional components.

This study also suggests that the required components for transcription/translation are likely exhausted within 24 h, resulting in a limited rate of transcription and translation. Since it has been suggested that different subpopulations of exosomes may possess distinct molecular characteristics (Willms et al., 2016), it is possible that only a small subset of exosomes possess the capacity for de novo protein synthesis. The newly synthetized proteins in exosomes are functionally active, suggestive of an appropriate protein conformation. It has been shown that exosomes contain not only the components of ribosomes, but also several molecular chaperones, such as Hsp60 and Hsp70. The ribosome itself has an important role in co-translational protein folding, for instance, it can promote the formation of secondary structures in newly formed proteins. The ribosome also acts as a platform for the association of chaperones that can assist with the appropriate folding of nascent proteins (Kramer et al., 2009). It is conceivable that these exosomes components could contribute to the stabilization of newly formed proteins. Physical confinement, as would be the case in the lumen of exosomes, can also have a stabilizing effect on the folding of proteins (Rao and Cruz, 2013). The possibility, however, cannot be ruled out that many proteins might exhibit inappropriate conformation or mis-folding. These could still have important biological implications, as demonstrated with the recent unraveling of unexpected features of the “dark proteome” (Perdigao et al., 2015), which suggested that proteins with unknown structure or intrinsically disordered regions may have important physiological functions.

A meticulous and quantitative identification of the proteins naturally synthetized in exosomes is necessary to fully appreciate the biological significance of this process. It is clear, however, that this could have significant impact in re-evaluating the understanding of eukaryotic biology. Recent studies suggest that cells can selectively incorporate mRNAs into exosomes (Raposo and Stoorvogel, 2013). This raises the possibility that mRNAs selectively packaged into exosomes could be translated into proteins whose expression is repressed in their cell of origin, as shown in this study as a proof of concept. The identification of newly synthetized proteins in exosomes up to one month after translation, suggests that exosomes-mediated production of proteins could lead to a significantly increased protein half-life, possibly due to lower levels of protein degradation enzymes.

Taken together, these results collectively demonstrate exosomes possess previously unappreciated biological activity with a potentially profound impact on body homeostasis and tissue pathogenesis. One could speculate that growth factor gradients could play a role on the systemic tropism of exosomes in the body. A disruption of the naturally occurring pattern of growth factor production could have immediate consequences on both the redistribution and delivery patterns of exosomes. These patterns of response could have potential implications in determining cell-cell communication, particularly between distant body sites. The fact that they can change their patterns of protein expression in response to these extracellular cues would suggest that exosomes could act as the primary responders in tissue injury. In conclusion, these findings provide a novel insight into the basic biology of exosomes and inform on their biological functions in organism homeostasis and their potential impact in disease states.

### I. LIPID-BASED NANOPARTICLES

In some embodiments, a lipid-based nanoparticle is a liposomes, an exosomes, lipid preparations, or another lipid-based nanoparticle, such as a lipid-based vesicle (e.g., a DOTAP:cholesterol vesicle). Lipid-based nanoparticles may be positively charged, negatively charged or neutral. Lipid-based nanoparticles may comprise the necessary components to allow for transcription and translation, signal transduction, chemotaxis, or other cellular functions.

A. Liposomes

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes, and multivesicular liposomes. Liposomes provided herein may be positively charged, negatively charged, or neutrally charged. In certain embodiments, the liposomes are neutral in charge.

A multilamellar liposome has multiple lipid layers separated by aqueous medium. Such liposomes form spontaneously when lipids comprising phospholipids are suspended in an excess of aqueous solution. The lipid components undergo self-rearrangement before the formation of closed structures and entrap water and dissolved solutes between the lipid bilayers. Lipophilic molecules or molecules with lipophilic regions may also dissolve in or associate with the lipid bilayer.

In specific aspects, a polypeptide, a nucleic acid, or a small molecule drug may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

A liposome used according to the present embodiments can be made by different methods, as would be known to one of ordinary skill in the art. For example, a phospholipid, such as for example the neutral phospholipid dioleoylphosphatidylcholine (DOPC), is dissolved in tert-butanol. The lipid(s) is then mixed with a polypeptide, nucleic acid, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 h, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

The dried lipids or lyophilized liposomes prepared as described above may be dehydrated and reconstituted in a solution of a protein or peptide and diluted to an appropriate concentration with a suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

Additional liposomes which may be useful with the present embodiments include cationic liposomes, for example, as described in WO02/100435A1, U.S. Pat. No. 5,962,016, U.S. Application 2004/0208921, WO03/015757A1, WO04029213A2, U.S. Pat. Nos. 5,030,453, and 6,680,068, all of which are hereby incorporated by reference in their entirety without disclaimer.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040, each incorporated herein by reference.

In certain embodiments, the lipid based nanoparticle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanoparticles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes. Phospholipids may be from natural or synthetic sources. Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

B. Exosomes

The terms “microvesicle” and “exosomes,” as used herein, refer to a membranous particle having a diameter (or largest dimension where the particles is not spheroid) of between about 10 nm to about 5000 nm, more typically between 30 nm and 1000 nm, and most typically between about 50 nm and 750 nm, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

Exosomes may also be isolated from tissue samples, such as surgical samples, biopsy samples, tissues, feces, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes contemplated herein are preferably isolated from body fluid in a physiologically acceptable solution, for example, buffered saline, growth medium, various aqueous medium, etc.

Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One preferred method is differential centrifugation from body fluids or cell culture supernatants. Exemplary methods for isolation of exosomes are described in (Losche et al., 2004; Mesri and Altieri, 1998; Morel et al., 2004). Alternatively, exosomes may also be isolated via flow cytometry as described in (Combes et al., 1997).

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. Similarly, a commercial kit is available (EXOMIR™, Bioo Scientific) which allows removal of cells, platelets, and cellular debris on one microfilter and capturing of vesicles bigger than 30 nm on a second microfilter using positive pressure to drive the fluid. However, for this process, the exosomes are not recovered, their RNA content is directly extracted from the material caught on the second microfilter, which can then be used for PCR analysis. HPLC-based protocols could potentially allow one to obtain highly pure exosomes, though these processes require dedicated equipment and are difficult to scale up. A significant problem is that both blood and cell culture media contain large numbers of nanoparticles (some non-vesicular) in the same size range as exosomes. For example, some miRNAs may be contained within extracellular protein complexes rather than exosomes; however, treatment with protease (e.g., proteinase K) can be performed to eliminate any possible contamination with “extraexosomal” protein.

In another embodiment, cancer cell-derived exosomes may be captured by techniques commonly used to enrich a sample for exosomes, such as those involving immunospecific interactions (e.g., immunomagnetic capture). Immunomagnetic capture, also known as immunomagnetic cell separation, typically involves attaching antibodies directed to proteins found on a particular cell type to small paramagnetic beads. When the antibody-coated beads are mixed with a sample, such as blood, they attach to and surround the particular cell. The sample is then placed in a strong magnetic field, causing the beads to pellet to one side. After removing the blood, captured cells are retained with the beads. Many variations of this general method are well-known in the art and suitable for use to isolate exosomes. In one example, the exosomes may be attached to magnetic beads (e.g., aldehyde/sulphate beads) and then an antibody is added to the mixture to recognize an epitope on the surface of the exosomes that are attached to the beads. Exemplary proteins that are known to be found on cancer cell-derived exosomes include ATP-binding cassette sub-family A member 6 (ABCA6), tetraspanin-4 (TSPAN4), SLIT and NTRK-like protein 4 (SLITRK4), putative protocadherin beta-18 (PCDHB18), myeloid cell surface antigen CD33 (CD33), and glypican-1 (GPC1). Cancer cell-derived exosomes may be isolated using, for example, antibodies or aptamers to one or more of these proteins.

As used herein, analysis includes any method that allows direct or indirect visualization of exosomes and may be in vivo or ex vivo. For example, analysis may include, but not limited to, ex vivo microscopic or cytometric detection and visualization of exosomes bound to a solid substrate, flow cytometry, fluorescent imaging, and the like. In an exemplary aspect, cancer cell-derived exosomes are detected using antibodies directed to one or more of ATP-binding cassette sub-family A member 6 (ABCA6), tetraspanin-4 (TSPAN4), SLIT and NTRK-like protein 4 (SLITRK4), putative protocadherin beta-18 (PCDHB18), myeloid cell surface antigen CD33 (CD33), glypican-1 (GPC1), Histone H2A type 2-A (HIST1H2AA), Histone H2A type 1-A (HIST1H1AA), Histone H3.3 (H3F3A), Histone H3.1 (HIST1H3A), Zinc finger protein 37 homolog (ZFP37), Laminin subunit beta-1 (LAMB1), Tubulointerstitial nephritis antigen-like (TINAGL1), Peroxiredeoxin-4 (PRDX4), Collagen alpha-2(IV) chain (COL4A2), Putative protein C3P1 (C3P1), Hemicentin-1 (HMCN1), Putative rhophilin-2-like protein (RHPN2P1), Ankyrin repeat domain-containing protein 62 (ANKRD62), Tripartite motif-containing protein 42 (TRIM42), Junction plakoglobin (JUP), Tubulin beta-2B chain (TUBB2B), Endoribonuclease Dicer (DICER1), E3 ubiquitin-protein ligase TRIM71 (TRIM71), Katanin p60 ATPase-containing subunit A-like 2 (KATNAL2), Protein S100-A6 (S100A6), 5′-nucleotidase domain-containing protein 3 (NT5DC3), Valine-tRNA ligase (VARS), Kazrin (KAZN), ELAV-like protein 4 (ELAVL4), RING finger protein 166 (RNF166), FERM and PDZ domain-containing protein 1 (FRMPD1), 78 kDa glucose-regulated protein (HSPA5), Trafficking protein particle complex subunit 6A (TRAPPC6A), Squalene monooxygenase (SQLE), Tumor susceptibility gene 101 protein (TSG101), Vacuolar protein sorting 28 homolog (VPS28), Prostaglandin F2 receptor negative regulator (PTGFRN), Isobutyryl-CoA dehydrogenase, mitochondrial (ACAD8), 26S protease regulatory subunit 6B (PSMC4), Elongation factor 1-gamma (EEF1G), Titin (TTN), Tyrosine-protein phosphatase type 13 (PTPN13), Triosephosphate isomerase (TPII), or Carboxypeptidase E (CPE) and subsequently bound to a solid substrate and/or visualized using microscopic or cytometric detection.

It should be noted that not all proteins expressing in a cell are found in exosomes secreted by that cell (see FIG. 11). For example, calnexin, GM130, and LAMP-2 are all proteins expressed in MCF-7 cells but not found in exosomes secreted by MCF-7 cells (Baietti et al., 2012). As another example, one study found that 190/190 pancreatic ductal adenocarcinoma patients had higher levels of GPC1+ exosomes than healthy controls (Melo et al., 2015, which is incorporated herein by reference in its entirety). Notably, only 2.3% of healthy controls, on average, had GPC1+ exosomes.

1. Exemplary Protocol for Collecting Exosomes from Cell Culture

On Day 1, seed enough cells (e.g., about five million cells) in T225 flasks in media containing 10% FBS so that the next day the cells will be about 70% confluent. On Day 2, aspirate the media on the cells, wash the cells twice with PBS, and then add 25-30 mL base media (i.e., no PenStrep or FBS) to the cells. Incubate the cells for 24-48 hours. A 48 hour incubation is preferred, but some cells lines are more sensitive to serum-free media and so the incubation time should be reduced to 24 hours. Note that FBS contains exosomes that will heavily skew NanoSight results.

On Day 3/4, collect the media and centrifuge at room temperature for five minutes at 800×g to pellet dead cells and large debris. Transfer the supernatant to new conical tubes and centrifuge the media again for 10 minutes at 2000×g to remove other large debris and large vesicles. Pass the media through a 0.2 μm filter and then aliquot into ultracentrifuge tubes (e.g., 25×89 mm Beckman Ultra-Clear) using 35 mL per tube. If the volume of media per tube is less than 35 mL, fill the remainder of the tube with PBS to reach 35 mL. Ultracentrifuge the media for 2-4 hours at 28,000 rpm at 4° C. using a SW 32 Ti rotor (k-factor 266.7, RCF max 133,907). Carefully aspirate the supernatant until there is roughly 1-inch of liquid remaining. Tilt the tube and allow remaining media to slowly enter aspirator pipette. If desired, the exosomes pellet can be resuspended in PBS and the ultracentrifugation at 28,000 rpm repeated for 1-2 hours to further purify the population of exosomes.

Finally, resuspend the exosomes pellet in 210 μL PBS. If there are multiple ultracentrifuge tubes for each sample, use the same 210 μL PBS to serially resuspend each exosomes pellet. For each sample, take 10 μL and add to 990 μL H₂O to use for nanoparticle tracking analysis. Use the remaining 200 μL exosomes-containing suspension for downstream processes or immediately store at −80° C.

2. Exemplary Protocol for Extracting Exosomes from Serum Samples

First, allow serum samples to thaw on ice. Then, dilute 250 μL of cell-free serum samples in 11 mL PBS; filter through a 0.2 μm pore filter. Ultracentrifuge the diluted sample at 150,000×g overnight at 4° C. The following day, carefully discard the supernatant and wash the exosomes pellet in 11 mL PBS. Perform a second round of ultracentrifugation at 150,000×g at 4° C. for 2 hours. Finally, carefully discard the supernatant and resuspend the exosomes pellet in 100 μL PBS for analysis.

C. Exemplary Protocol for Electroporation of Exosomes and Liposomes

Mix 1×10⁸ exosomes (measured by NanoSight analysis) or 100 nm liposomes (e.g., purchased from Encapsula Nano Sciences) and 1 μg of siRNA (Qiagen) or shRNA in 400 μL of electroporation buffer (1.15 mM potassium phosphate, pH 7.2, 25 mM potassium chloride, 21% Optiprep). Electroporate the exosomes or liposomes using a 4 mm cuvette (see, e.g., Alvarez-Erviti et al., 2011; El-Andaloussi et al., 2012). After electroporation, treat the exosomes or liposomes with protease-free RNAse followed by addition of 10× concentrated RNase inhibitor. Finally, wash the exosomes or liposomes with PBS under ultracentrifugation methods, as described above.

### II. DIAGNOSIS, PROGNOSIS, AND TREATMENT OF DISEASES

Certain aspects of the present invention provide for treating a patient with exosomes that express or comprise a therapeutic agent or a diagnostic agent. A “therapeutic agent” as used herein is an atom, molecule, or compound that is useful in the treatment of cancer or other conditions. Examples of therapeutic agents include, but are not limited to, drugs, chemotherapeutic agents, therapeutic antibodies and antibody fragments, toxins, radioisotopes, enzymes, nucleases, hormones, immunomodulators, antisense oligonucleotides, chelators, boron compounds, photoactive agents, and dyes. A “diagnostic agent” as used herein is an atom, molecule, or compound that is useful in diagnosing, detecting or visualizing a disease. According to the embodiments described herein, diagnostic agents may include, but are not limited to, radioactive substances (e.g., radioisotopes, radionuclides, radiolabels or radiotracers), dyes, contrast agents, fluorescent compounds or molecules, bioluminescent compounds or molecules, enzymes and enhancing agents (e.g., paramagnetic ions).

In some aspects, a therapeutic recombinant protein may be a protein having an activity that has been lost in a cell of the patient, a protein having a desired enzymatic activity, a protein having a desired inhibitory activity, etc. For example, the protein may be a transcription factor, an enzyme, a proteinaceous toxin, an antibody, a monoclonal antibody, etc. The monoclonal antibody may specifically or selectively bind to an intracellular antigen. The monoclonal antibody may inhibit the function of the intracellular antigen and/or disrupt a protein-protein interaction. Other aspects of the present invention provide for diagnosing a disease based on the presence of cancer cell-derived exosomes in a patient sample.

As exosomes are known to comprise the machinery necessary to complete mRNA transcription and protein translation (see PCT/US2014/068630, which is incorporated herein by reference in its entirety), mRNA or DNA nucleic acids encoding a therapeutic protein may be transfected into exosomes. Alternatively, the therapeutic protein itself may be electroporated into the exosomes or incorporated directly into a liposome. Exemplary therapeutic proteins include, but are not limited to, a tumor suppressor protein, peptides, a wild type protein counterparts of a mutant protein, a DNA repair protein, a proteolytic enzyme, proteinaceous toxin, a protein that can inhibit the activity of an intracellular protein, a protein that can activate the activity of an intracellular protein, or any protein whose loss of function needs to be reconstituted. Specific examples of exemplary therapeutic proteins include 123F2, Abcb4, Abcc1, Abcg2, Actb, Ada, Ahr, Akt, Akt1, Akt2, Akt3, Amhr2, Anxa7, Apc, Ar, Atm, Axin2, B2m, Bard1, Bcl2l1, Becn1, Bhlhal5, Bin1, Blm, Braf, Brca1, Brca2, Brca3, Braf, Brcata, Brinp3, Brip1, Bub1b, Bwscr1a, Cadm3, Casc1, Casp3, Casp7, Casp8, Cav1, Ccam, Ccnd1, Ccr4, Ccs1, Cd28, Cdc25a, Cd95, Cdh1, Cdkn1a, Cdkn1b, Cdkn2a, Cdkn2b, Cdkn2c, Cftr, Chek1, Chek2, Crcs1, Cres10, Crcs11, Crcs2, Crcs3, Crcs4, Crcs5, Crcs6, Crcs7, Crcs8, Crcs9, Ctnnb1, Cts1, Cyp1a1, Cyp2a6, Cyp2b2, Cyld, Dcc, Dkc1, Dicer1, Dmtf1, Dnmt1, Dpc4, E2f1, Eaf2, Eef1a1, Egfr, Egfr4, Erbb2, Erbb4, Ercc2, Ercc6, Ercc8, Errfi1, Esr1, Etv4, Fas1g, Fbxo10, Fcc, Fgfr3, Fntb, Foxm1, Foxn1, Fus1, Fzd6, Fzd7, Fzr1, Gadd45a, Gast, Gnai2, Gpc1, Gpr124, Gpr87, Gprc5a, Gprc5d, Grb2, Gstm1, Gstm5, Gstp1, Gstt1, H19, H2afx, Hck, Lims1, Hdac, Hexa, Hic1, Hin1, Hmmr, Hnpcc8, Hprt, Hras, Htatip2, Il1b, Il10, Il2, Il6, Il8rb Inha, Itgav, Jun, Jak3, Kit, Klf4, Kras, Kras2, Kras2b, Lig1, Lig4, Lkb1, Lmo7, Lncr1, Lncr2, Lncr3, Lncr4, Ltbp4, Luca1, Luca2, Lyz2, Lzts1, Mad111, Mad211, Madr2/Jv18, Mapk14, Mcc, Mcm4, Men1, Men2, Met, Mgat5, Mif, Mlh1, Mlh3, Mmac1, Mmp8, Mnt, Mpo, Msh2, Msh3, Msh6, Msmb, Mthfr, Mts1, Mutyh, Myh11, Nat2, Nbn, Ncoa3, Neil1, Nf1, Nf2, Nfe2l1, Nhej1, Nkx2-1, Nkx2-9, Nkx3-1, Nprl2, Nqo1, Nras, Nudt1, Ogg1, Oxgr1, p16, p19, p21, p27, p27mt, p57, p14ARF, Palb2, Park2, Pggt1b, Pgr, Pi3k, Pik3ca, Piwil2, Pl6, Pla2g2a, Plg, Plk3, Pms1, Pms2, Pold1, Pole, Ppard, Pparg, Ppfia2, Ppm1d, Prdm2, Prdx1, Prkar1a, Ptch, Pten, Prom1, Psca, Ptch1, Ptf1a, Ptger2, Ptpn13, Ptprj, Rara, Rad51, Rassf1, Rb, Rb1, Rb1cc1, Rb12, Recg14, Ret, Rgs5, Rhoc, Rint1, Robo1, Rpl38, S100a4, SCGB1A1, Skp2, Smad2, Smad3, Smad4, Smarcb1, Smo, Snx25, Spata13, Srpx, Ssic1, Sstr2, Sstr5, Stat3, St5, St7, St14, Stk11, Suds3, Tap1, Tbx21, Terc, Tnf, Tp53, Tp73, Trpm5, Tsc2, Tsc1, Vh1, Wrn, Wt1, Wt2, Xrcc1, Xrcc5, Xrcc6, and Zac1.

One specific type of protein that it may be desirable to introduce into the intracellular space of a diseased cell is an antibody (e.g., a monoclonal antibody). Such an antibody may disrupt the function of an intracellular protein and/or disrupt an intracellular protein-protein interaction. Exemplary targets of such monoclonal antibodies include, but are not limited to, proteins involved in the RNAi pathway, telomerase, transcription factors that control disease processes, kinases, phosphatases, proteins required for DNA synthesis, protein required for protein translation. Specific examples of exemplary therapeutic antibody targets include proteins encoded by the following genes: Dicer, Ago1, Ago2, Trbp, Ras, raf, wnt, btk, Bcl-2, Akt, Sis, src, Notch, Stathmin, mdm2, abl, hTERT, c-fos, c-jun, c-myc, erbB, HER2/Neu, HER3, VEGFR, PDGFR, c-kit, c-met, c-ret, flt3, API, AML1, axl, alk, fins, fps, gip, lck, Stat, Hox, MLM, PRAD-I, and trk. In addition to monoclonal antibodies, any antigen binding fragment there of, such as a scFv, a Fab fragment, a Fab′, a F(ab′)2, a Fv, a peptibody, a diabody, a triabody, or a minibody, is also contemplated. Any such antibodies or antibody fragment may be either glycosylated or aglycosylated.

As exosomes are known to comprise DICER and active RNA processing RISC complex (see PCT Publn. WO 2014/152622, which is incorporated herein by reference in its entirety), shRNA transfected into exosomes can mature into RISC-complex bound siRNA with the exosomes themselves. Alternatively, mature siRNA can itself be transfected into exosomes or liposomes. Thus, by way of example, the following are classes of possible target genes that may be used in the methods of the present invention to modulate or attenuate target gene expression: wild-type or mutant versions of developmental genes (e.g., adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors), tumor suppressor genes (e.g., APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide), pro-apoptotic genes (e.g., CD95, caspase-3, Bax, Bag-1, CRADD, TSSC3, bax, hid, Bak, MKP-7, PARP, bad, bcl-2, MST1, bbc3, Sax, BIK, and BID), cytokines (e.g., GM-CSF, G-CSF, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32 IFN-α, IFN-β, IFN-γ, MIP-1α, MIP-10, TGF-β, TNF-α, TNF-β, PDGF, and mda7), oncogenes (e.g., ABLI, BLC1, BCL6, CBFA1, CBL, CSFIR, ERBA, ERBB, EBRB2, ETS1, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3 and YES), and enzymes (e.g., ACP desaturases and hycroxylases, ADP-glucose pyrophorylases, ATPases, alcohol dehycrogenases, amylases, amyloglucosidases, catalases, cellulases, cyclooxygenases, decarboxylases, dextrinases, esterases, DNA and RNA polymerases, galactosidases, glucanases, glucose oxidases, GTPases, helicases, hemicellulases, integrases, invertases, isomersases, kinases, lactases, lipases, lipoxygenases, lysozymes, nucleases, pectinesterases, peroxidases, phosphatases, phospholipases, phosphorylases, polygalacturonases, proteinases and peptideases, pullanases, recombinases, reverse transcriptases, topoisomerases, xylanases). In some cases, sh/siRNA may be designed to specifically target a mutant version of a gene expressed in a cancer cell while not affecting the expression of the corresponding wild-type version. In fact, any inhibitory nucleic acid that can be applied in the compositions and methods of the present invention if such inhibitory nucleic acid has been found by any source to be a validated downregulator of a protein of interest.

In designing RNAi there are several factors that need to be considered, such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80%, 85%, 90%, 95%, 98%, or even 100% identity between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

Exosomes may also be engineered to comprise a gene editing system, such as a CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The CRISPR system in exosomes engineered to comprise such a system may function to edit the genomic DNA inside a target cell, or the system may edit the DNA inside the exosomes itself.

In addition to protein- and nucleic acid-based therapeutics, exosomes may be used to deliver small molecule drugs, either alone or in combination with any protein- or nucleic acid-based therapeutic. Exemplary small molecule drugs that are contemplated for use in the present embodiments include, but are not limited to, toxins, chemotherapeutic agents, agents that inhibit the activity of an intracellular protein, agents that activate the activity of intracellular proteins, agents for the prevention of restenosis, agents for treating renal disease, agents used for intermittent claudication, agents used in the treatment of hypotension and shock, angiotensin converting enzyme inhibitors, antianginal agents, anti-arrhythmics, anti-hypertensive agents, antiotensin ii receptor antagonists, antiplatelet drugs, b-blockers b1 selective, beta blocking agents, botanical product for cardiovascular indication, calcium channel blockers, cardiovascular/diagnostics, central alpha-2 agonists, coronary vasodilators, diuretics and renal tubule inhibitors, neutral endopeptidase/angiotensin converting enzyme inhibitors, peripheral vasodilators, potassium channel openers, potassium salts, anticonvulsants, antiemetics, antinauseants, anti-parkinson agents, antispasticity agents, cerebral stimulants, agents that can be applied in the treatment of trauma, agents that can be applied in the treatment of Alzheimer disease or dementia, agents that can be applied in the treatment of migraine, agents that can be applied in the treatment of neurodegenerative diseases, agents that can be applied in the treatment of kaposi's sarcoma, agents that can be applied in the treatment of AIDS, cancer chemotherapeutic agents, agents that can be applied in the treatment of immune disorders, agents that can be applied in the treatment of psychiatric disorders, analgesics, epidural and intrathecal anesthetic agents, general, local, regional neuromuscular blocking agents sedatives, preanesthetic adrenal/acth, anabolic steroids, agents that can be applied in the treatment of diabetes, dopamine agonists, growth hormone and analogs, hyperglycemic agents, hypoglycemic agents, oral insulins, large volume parenterals (lvps), lipid-altering agents, metabolic studies and inborn errors of metabolism, nutrients/amino acids, nutritional lvps, obesity drugs (anorectics), somatostatin, thyroid agents, vasopressin, vitamins, corticosteroids, mucolytic agents, pulmonary anti-inflammatory agents, pulmonary surfactants, antacids, anticholinergics, antidiarrheals, antiemetics, cholelitholytic agents, inflammatory bowel disease agents, irritable bowel syndrome agents, liver agents, metal chelators, miscellaneous gastric secretory agents, pancreatitis agents, pancreatic enzymes, prostaglandins, prostaglandins, proton pump inhibitors, sclerosing agents, sucralfate, anti-progestins, contraceptives, oral contraceptives, not oral dopamine agonists, estrogens, gonadotropins, GNRH agonists, GHRH antagonists, oxytocics, progestins, uterine-acting agents, anti-anemia drugs, anticoagulants, antifibrinolytics, antiplatelet agents, antithrombin drugs, coagulants, fibrinolytics, hematology, heparin inhibitors, metal chelators, prostaglandins, vitamin K, anti-androgens, aminoglycosides, antibacterial agents, sulfonamides, cephalosporins, clindamycins, dermatologics, detergents, erythromycins, anthelmintic agents, antifungal agents, antimalarials, antimycobacterial agents, antiparasitic agents, antiprotozoal agents, antitrichomonads, antituberculosis agents, immunomodulators, immunostimulatory agents, macrolides, antiparasitic agents, corticosteroids, cyclooxygenase inhibitors, enzyme blockers, immunomodulators for rheumatic diseases, metalloproteinase inhibitors, nonsteroidal anti-inflammatory agents, analgesics, antipyretics, alpha adrenergic agonists/blockers, antibiotics, antivirals, beta adrenergic blockers, carbonic anhydrase inhibitors, corticosteroids, immune system regulators, mast cell inhibitors, nonsteroidal anti-inflammatory agents, and prostaglandins.

Exosomes may also be used to deliver diagnostic agents. Exemplary diagnostic agents include, but are not limited to, magnetic resonance image enhancement agents, positron emission tomography products, radioactive diagnostic agents, radioactive therapeutic agents, radio-opaque contrast agents, radiopharmaceuticals, ultrasound imaging agents, and angiographic diagnostic agents.

The term “subject” as used herein refers to any individual or patient to which the subject methods are performed. Generally the subject is human, although as will be appreciated by those in the art, the subject may be an animal. Thus other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of chemotherapy, immunotherapy, or radiotherapy, performance of surgery, or any combination thereof.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The terms “contacted” and “exposed,” when applied to a cell, are used herein to describe the process by which a therapeutic agent are delivered to a target cell or are placed in direct juxtaposition with the target cell. To achieve cell killing, for example, one or more agents are delivered to a cell in an amount effective to kill the cell or prevent it from dividing.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Treatment outcomes can be predicted and monitored and/or patients benefiting from such treatments can be identified or selected via the methods described herein.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the treatment of disease, the appropriate dosage of a therapeutic composition will depend on the type of disease to be treated, as defined above, the severity and course of the disease, the patient's clinical history and response to the agent, and the discretion of the attending physician. The agent is suitably administered to the patient at one time or over a series of treatments.

Therapeutic and prophylactic methods and compositions can be provided in a combined amount effective to achieve the desired effect. A tissue, tumor, or cell can be contacted with one or more compositions or pharmacological formulation(s) comprising one or more of the agents, or by contacting the tissue, tumor, and/or cell with two or more distinct compositions or formulations. Also, it is contemplated that such a combination therapy can be used in conjunction with chemotherapy, radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

A first anti-cancer treatment (e.g., exosomes that express a recombinant protein or with a recombinant protein isolated from exosomes) may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below a first anti-cancer therapy is “A” and a second anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaII); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolvsaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO*, is an anti-PD-1 antibody described in WO2006/121168. Pembrolizumab, also known as MK-3475, Merck 3475, lambrolizumab, KEYTRUDA*, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 900%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering (Park, Rosenberg et al. 2011). Isolation and transfer of tumor specific T cells has been shown to be successful in treating melanoma. Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors (Jena, Dotti et al. 2010).

In one embodiment, the present application provides for a combination therapy for the treatment of cancer wherein the combination therapy comprises adoptive T-cell therapy and a checkpoint inhibitor. In one aspect, the adoptive T-cell therapy comprises autologous and/or allogenic T cells. In another aspect, the autologous and/or allogenic T cells are targeted against tumor antigens.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

### III. PHARMACEUTICAL COMPOSITIONS

It is contemplated that exosomes that express or comprise a therapeutic protein, inhibitory RNA, and/or small molecule drug can be administered systemically or locally to inhibit tumor cell growth and, most preferably, to kill cancer cells in cancer patients with locally advanced or metastatic cancers. They can be administered intravenously, intrathecally, and/or intraperitoneally. They can be administered alone or in combination with anti-proliferative drugs. In one embodiment, they are administered to reduce the cancer load in the patient prior to surgery or other procedures. Alternatively, they can be administered after surgery to ensure that any remaining cancer (e.g., cancer that the surgery failed to eliminate) does not survive.

It is not intended that the present invention be limited by the particular nature of the therapeutic preparation. For example, such compositions can be provided in formulations together with physiologically tolerable liquid, gel, solid carriers, diluents, or excipients. These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particular requirements of individual subjects.

Where clinical applications are contemplated, it may be necessary to prepare pharmaceutical compositions comprising recombinant proteins and/or exosomes in a form appropriate for the intended application. Generally, pharmaceutical compositions may comprise an effective amount of one or more recombinant proteins and/or exosomes or additional agents dissolved or dispersed in a pharmaceutically acceptable carrier. The phrases “pharmaceutical or pharmacologically acceptable” refers to molecular entities and compositions that do not produce an adverse, allergic, or other untoward reaction when administered to an animal, such as, for example, a human, as appropriate. The preparation of a pharmaceutical composition comprising a recombinant protein and/or exosomes as disclosed herein, or additional active ingredient will be known to those of skill in the art in light of the present disclosure, as exemplified by Remington's Pharmaceutical Sciences, 18th Ed., 1990, incorporated herein by reference. Moreover, for animal (e.g., human) administration, it will be understood that preparations should meet sterility, pyrogenicity, general safety, and purity standards as required by the FDA Office of Biological Standards.

Further in accordance with certain aspects of the present invention, the composition suitable for administration may be provided in a pharmaceutically acceptable carrier with or without an inert diluent. As used herein, “pharmaceutically acceptable carrier” includes any and all aqueous solvents (e.g., water, alcoholic/aqueous solutions, ethanol, saline solutions, parenteral vehicles, such as sodium chloride, Ringer's dextrose, etc.), non-aqueous solvents (e.g., fats, oils, polyol (for example, glycerol, propylene glycol, and liquid polyethylene glycol, and the like), vegetable oil, and injectable organic esters, such as ethyloleate), lipids, liposomes, dispersion media, coatings (e.g., lecithin), surfactants, antioxidants, preservatives (e.g., antibacterial or antifungal agents, anti-oxidants, chelating agents, inert gases, parabens (e.g., methylparabens, propylparabens), chlorobutanol, phenol, sorbic acid, thimerosal or combinations thereof), isotonic agents (e.g., sugars and sodium chloride), absorption delaying agents (e.g., aluminum monostearate and gelatin), salts, drugs, drug stabilizers, gels, resins, fillers, binders, excipients, disintegration agents, lubricants, sweetening agents, flavoring agents, dyes, fluid and nutrient replenishers, such like materials and combinations thereof, as would be known to one of ordinary skill in the art. The carrier should be assimilable and includes liquid, semi-solid, i.e., pastes, or solid carriers. In addition, if desired, the compositions may contain minor amounts of auxiliary substances, such as wetting or emulsifying agents, stabilizing agents, or pH buffering agents. The pH and exact concentration of the various components in a pharmaceutical composition are adjusted according to well-known parameters. The proper fluidity can be maintained, for example, by the use of a coating, such as lecithin, by the maintenance of the required particle size in the case of dispersion, and by the use of surfactants.

A pharmaceutically acceptable carrier is particularly formulated for administration to a human, although in certain embodiments it may be desirable to use a pharmaceutically acceptable carrier that is formulated for administration to a non-human animal but that would not be acceptable (e.g., due to governmental regulations) for administration to a human. Except insofar as any conventional carrier is incompatible with the active ingredient (e.g., detrimental to the recipient or to the therapeutic effectiveness of a composition contained therein), its use in the therapeutic or pharmaceutical compositions is contemplated. In accordance with certain aspects of the present invention, the composition is combined with the carrier in any convenient and practical manner, i.e., by solution, suspension, emulsification, admixture, encapsulation, absorption, and the like. Such procedures are routine for those skilled in the art.

Certain embodiments of the present invention may comprise different types of carriers depending on whether it is to be administered in solid, liquid, or aerosol form, and whether it needs to be sterile for the route of administration, such as injection. The compositions can be administered intravenously, intradermally, transdermally, intrathecally, intraarterially, intraperitoneally, intranasally, intravaginally, intrarectally, intramuscularly, subcutaneously, mucosally, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference).

The active compounds can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The therapeutics may be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid and the like. Salts formed with the free carboxyl groups can also be derived from inorganic bases, such as, for example, sodium, potassium, ammonium, calcium, or ferric hydroxides: or such organic bases as isopropylamine, trimethylamine, histidine, or procaine and the like. Upon formulation, solutions will be administered in a manner compatible with the dosage formulation and in such amount as is therapeutically effective. The formulations are easily administered in a variety of dosage forms, such as formulated for parenteral administrations, such as injectable solutions, or aerosols for delivery to the lungs, or formulated for alimentary administrations, such as drug release capsules and the like.

In a specific embodiment of the present invention, the composition is combined or mixed thoroughly with a semi-solid or solid carrier. The mixing can be carried out in any convenient manner, such as grinding. Stabilizing agents can be also added in the mixing process in order to protect the composition from loss of therapeutic activity, i.e., denaturation in the stomach. Examples of stabilizers for use in a composition include buffers, amino acids, such as glycine and lysine, carbohydrates, such as dextrose, mannose, galactose, fructose, lactose, sucrose, maltose, sorbitol, mannitol, etc.

In further embodiments, the present invention may concern the use of a pharmaceutical lipid vehicle composition comprising one or more lipids and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Of course, compounds other than those specifically described herein that are understood by one of skill in the art as lipids are also encompassed by the compositions and methods.

One of ordinary skill in the art would be familiar with the range of techniques that can be employed for dispersing a composition in a lipid vehicle. For example, the therapeutic agent may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present invention administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The actual dosage amount of a composition administered to an animal patient can be determined by physical and physiological factors, such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors, such as solubility, bioavailability, biological half-life, route of administration, product shelf life, as well as other pharmacological considerations, will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above.

### IV. NUCLEIC ACIDS AND VECTORS

In certain aspects of the invention, nucleic acid sequences encoding a therapeutic protein or a fusion protein containing a therapeutic protein may be disclosed. Depending on which expression system is used, nucleic acid sequences can be selected based on conventional methods. For example, the respective genes or variants thereof may be codon optimized for expression in a certain system. Various vectors may be also used to express the protein of interest. Exemplary vectors include, but are not limited, plasmid vectors, viral vectors, transposon, or liposome-based vectors.

### V. RECOMBINANT PROTEINS, INHIBITORY RNAS, AND GENE EDITING SYSTEMS

A. Recombinant Proteins

Some embodiments concern recombinant proteins and polypeptides. Particular embodiments concern a recombinant protein or polypeptide that exhibits at least one therapeutic activity. In some embodiments, a recombinant protein or polypeptide may be a therapeutic antibody. In some aspects, a therapeutic antibody may be an antibody that specifically or selectively binds to an intracellular protein. In further aspects, the protein or polypeptide may be modified to increase serum stability. Thus, when the present application refers to the function or activity of “modified protein” or a “modified polypeptide,” one of ordinary skill in the art would understand that this includes, for example, a protein or polypeptide that possesses an additional advantage over the unmodified protein or polypeptide. It is specifically contemplated that embodiments concerning a “modified protein” may be implemented with respect to a “modified polypeptide,” and vice versa.

Recombinant proteins may possess deletions and/or substitutions of amino acids; thus, a protein with a deletion, a protein with a substitution, and a protein with a deletion and a substitution are modified proteins. In some embodiments, these proteins may further include insertions or added amino acids, such as with fusion proteins or proteins with linkers, for example. A “modified deleted protein” lacks one or more residues of the native protein, but may possess the specificity and/or activity of the native protein. A “modified deleted protein” may also have reduced immunogenicity or antigenicity. An example of a modified deleted protein is one that has an amino acid residue deleted from at least one antigenic region that is, a region of the protein determined to be antigenic in a particular organism, such as the type of organism that may be administered the modified protein.

Substitution or replacement variants typically contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

In addition to a deletion or substitution, a modified protein may possess an insertion of residues, which typically involves the addition of at least one residue in the polypeptide. This may include the insertion of a targeting peptide or polypeptide or simply a single residue. Terminal additions, called fusion proteins, are discussed below.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a control polypeptide are included, provided the biological activity of the protein is maintained. A recombinant protein may be biologically functionally equivalent to its native counterpart in certain aspects.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

As used herein, a protein or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide,” and “peptide are used interchangeably herein.

As used herein, an “amino acid residue” refers to any naturally occurring amino acid, any amino acid derivative, or any amino acid mimic known in the art. In certain embodiments, the residues of the protein or peptide are sequential, without any non-amino acids interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid.

Certain embodiments of the present invention concern fusion proteins. These molecules may have a therapeutic protein linked at the N- or C-terminus to a heterologous domain. For example, fusions may also employ leader sequences from other species to permit the recombinant expression of a protein in a heterologous host. Another useful fusion includes the addition of a protein affinity tag, such as a serum albumin affinity tag or six histidine residues, or an immunologically active domain, such as an antibody epitope, preferably cleavable, to facilitate purification of the fusion protein. Non-limiting affinity tags include polyhistidine, chitin binding protein (CBP), maltose binding protein (MBP), and glutathione-S-transferase (GST).

In a particular embodiment, the therapeutic protein may be linked to a peptide that increases the in vivo half-life, such as an XTEN polypeptide (Schellenberger et al., 2009), IgG Fc domain, albumin, or albumin binding peptide.

Methods of generating fusion proteins are well known to those of skill in the art. Such proteins can be produced, for example, by de novo synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that is spliced between the polypeptides connected in tandem. The linker would be of sufficient length to allow proper folding of the resulting fusion protein.

B. Inhibitory RNAs

siNA (e.g., siRNA) are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Within a siNA, the components of a nucleic acid need not be of the same type or homogenous throughout (e.g., a siNA may comprise a nucleotide and a nucleic acid or nucleotide analog). Typically, siNA form a double-stranded structure; the double-stranded structure may result from two separate nucleic acids that are partially or completely complementary. In certain embodiments of the present invention, the siNA may comprise only a single nucleic acid (polynucleotide) or nucleic acid analog and form a double-stranded structure by complementing with itself (e.g., forming a hairpin loop). The double-stranded structure of the siNA may comprise 16, 20, 25, 30, 35, 40, 45, 50, 60, 65, 70, 75, 80, 85, 90, 100, 150, 200, 250, 300, 350, 400, 450, 500 or more contiguous nucleobases, including all ranges therein. The siNA may comprise 17 to 35 contiguous nucleobases, more preferably 18 to 30 contiguous nucleobases, more preferably 19 to 25 nucleobases, more preferably 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

Agents of the present invention useful for practicing the methods of the present invention include, but are not limited to siRNAs. Typically, introduction of double-stranded RNA (dsRNA), which may alternatively be referred to herein as small interfering RNA (siRNA), induces potent and specific gene silencing, a phenomena called RNA interference or RNAi. RNA interference has been referred to as “cosuppression,” “post-transcriptional gene silencing,” “sense suppression,” and “quelling.” RNAi is an attractive biotechnological tool because it provides a means for knocking out the activity of specific genes.

In designing RNAi there are several factors that need to be considered, such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80%, 85%, 90%, 95%, 98%, or even 1000% identity between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

In addition, the size of the siRNA is an important consideration. In some embodiments, the present invention relates to siRNA molecules that include at least about 19-25 nucleotides and are able to modulate gene expression. In the context of the present invention, the siRNA is preferably less than 500, 200, 100, 50, or 25 nucleotides in length. More preferably, the siRNA is from about 19 nucleotides to about 25 nucleotides in length.

A target gene generally means a polynucleotide comprising a region that encodes a polypeptide, or a polynucleotide region that regulates replication, transcription, or translation or other processes important to expression of the polypeptide, or a polynucleotide comprising both a region that encodes a polypeptide and a region operably linked thereto that regulates expression. Any gene being expressed in a cell can be targeted. Preferably, a target gene is one involved in or associated with the progression of cellular activities important to disease or of particular interest as a research object.

siRNA can be obtained from commercial sources, natural sources, or can be synthesized using any of a number of techniques well-known to those of ordinary skill in the art. For example, one commercial source of predesigned siRNA is Ambion®, Austin, Tex. Another is Qiagen® (Valencia, Calif.). An inhibitory nucleic acid that can be applied in the compositions and methods of the present invention may be any nucleic acid sequence that has been found by any source to be a validated downregulator of a protein of interest. Without undue experimentation and using the disclosure of this invention, it is understood that additional siRNAs can be designed and used to practice the methods of the invention.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In certain aspects, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Application Publication 2004/0019001 and U.S. Pat. No. 6,673,611 (each of which is incorporated by reference in its entirety). Collectively, all such altered nucleic acids or RNAs described above are referred to as modified siRNAs.

C. Gene Editing Systems

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type 111 CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### VI. KITS AND DIAGNOSTICS

In various aspects of the invention, a kit is envisioned containing the necessary components to purify exosomes from a body fluid or tissue culture medium. In other aspects, a kit is envisioned containing the necessary components to isolate exosomes and transfect them with a therapeutic nucleic acid, therapeutic protein, or a nucleic acid encoding a therapeutic protein therein. The kit may comprise one or more sealed vials containing any of such components. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass. The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of purifying exosomes from a sample and transfecting a therapeutic nucleic acid therein, expressing a recombinant protein therein, or electroporating a recombinant protein therein.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

Cell Culture. MCF7, MDA-MB231, E10, HDF, and BJ human cell lines, as well as the NIH 3T3 murine cell line were cultured in DMEM with 10% FBS. The 4T1 and 67NR murine cell lines were cultured in RPMI with 10% FBS. MCF10A human mammary epithelial cell line was cultured in DMEM/F12 media with 5% Horse Serum, 20 ng/ml EGF, 0.5 mg/ml Hydrocortisone, 100 ng/ml Cholera Toxin, and 10 μg/ml Insulin. All cells originated from the American Type Culture Collection—ATCC.

Isolation and purification of exosomes. Exosomes were purified by differential centrifugation as described previously (Luga et al., 2012; Thery et al., 2006). Supernatant from cells cultured for 48 h were subjected to sequential centrifugation steps of 800 g and 2000 g. The resulting supernatant was filtered using a 0.2 μm filter. A pellet was recovered after ultracentrifugation in an SW40Ti swinging bucket rotor at 100,000 g for 3 h (Beckman-Coulter). Supernatant was removed and the pellet was re-suspended in PBS, followed by a second ultracentrifugation at 100,000 g for 3 h. The resulting pellet was analyzed for exosomes content. Exosomes used for RNA extraction were resuspended in 500 μL of Trizol; exosomes used for protein extraction were resuspended in Urea/SDS lysis buffer (8M Urea, 2.5% SDS, 5 μg/mL leupeptin, 1 μg/mL pepstatin, and 1 mM phenylmethylsulphonyl fluoride); and exosomes used for delivery to cells were re-suspended in serum-free DMEM culture medium. For other applications, isolated exosomes were processed as described in the remaining experimental procedures.

Imaging Flow Cytometry analysis (ImageStream). Exosomes were attached to 4 μm aldehyde/sulfate latex beads (Invitrogen, Carlsbad, Calif., USA) in NaCl 0.9% saline solution (B. Braun Medical Inc, Bethlehem, Pa., USA). The reaction was stopped with 100 mM glycine and 2% BSA in saline and blocked with 10% BSA with rotation at room temperature for 30 min. After washing in saline/2% BSA, bead-bound exosomes were centrifuged for 2 min at 10,000 rpm and incubated with 1:200 anti-CD63 (Santa Cruz), anti-CD9 (Abcam), anti-CD81 (Abcam), anti-CD82 (Abcam), and anti-FLOT1 (Santa Cruz) for 30 min rotating at 4° C. Beads were centrifuged for 2 min at 10,000 rpm, washed in saline/2% BSA and incubated with 1:400 Alexa-488 secondary antibodies (Life Technologies, NY 14072) for 30 min rotating at 4° C. After three washes the beads were resuspended in saline solution and analyzed on the ImageStream® (Merck Millipore). The image acquisition gain (%) was set using a positive sample, in order to avoid pixel saturation. Image processing was done using the IDEAS® (Merck Millipore) software. Gates were defined to exclude out-of-focus beads and select single beads. Alexa-488 positive bead gates were defined on the negative control sample. Percentage of positive beads is relative to the number of events analyzed per sample.

Immunogold Labeling and Electron Microscopy. Pelleted exosomes were fixed by re-suspending in 2.5% Glutaraldehyde in 0.1 M Phosphate buffer. Fixed specimens at an optimal concentration were placed onto a 300 mesh carbon/formvar coated grids and allowed to absorb to the formvar for a minimum of 1 minute. For immunogold staining the grids were placed into a blocking buffer for a block/permeabilization step for 1 h. Without rinsing, the grids were immediately placed into the primary antibody at the appropriate dilution overnight at 4° C. (polyclonal anti-GFP 1:10, Abcam). As controls, some grids were not exposed to the primary antibody. The next day all grids were rinsed with PBS and floated on drops of the appropriate secondary antibody attached with 10 nm gold particles (AURION, Hatfield, Pa.) for 2 h at room temperature. Grids were rinsed with PBS and placed in 2.5% Glutaraldehyde in 0.1 M Phosphate buffer for 15 minutes. After rinsing in PBS and distilled water, the grids were allowed to dry and stained for contrast using uranyl acetate. The samples were viewed with a Tecnai Bio Twin transmission electron microscope (FEI, Hillsboro, Oreg.) and images were taken with an AMT CCD Camera (Advanced Microscopy Techniques, Danvers, Mass.).

EGF stimulation of exosomes. Exosomes were collected from MBA-MB-231 cells as described above. 1-3×10⁹ exosomes were resuspended in 1 mL PBS and different concentrations of rEGF were added. Exosomes suspensions, with or without EGF, were incubated at 37° C. with 5% CO₂ for 15 minutes and then placed on ice. Three replicates were pooled and PBS was added to a total volume of 11 mL and stimulated exosomes were collected through ultracentrifugation in an SW40Ti swinging bucket rotor at 100,000 g for 3 h, as before. Protein extracts were collected from the pelleted stimulated exosomes in Urea/SDS lysis buffer (with 100 mM NaF and 1 mM NaOV₄) for immunoblot analysis or a Triton X-100 buffer (150 mM NaCl, 1% (v/v) Triton X-100, 10 mM Na2HPO4, 2 mM KH2PO4, pH 7.4, 50 mM 6-aminohexanoic acid, 10 mM EDTA, 5 mM N-ethylmaleimide, 5 mM benzamidine, 5 μg/mL leupeptin, 1 μg/mL pepstatin, 1 mM phenylmethylsulphonyl fluoride, 100 mM NaF, and 1 mM NaOV₄) for immunoprecipitation assays.

Protein Western Blot and Antibodies. Exosomes protein extracts were loaded according to a Bicinchoninic Acid (BCA) protein assay kit (Pierce, Thermo Fisher Scientific) onto acrylamide gels and transferred onto PVDF membranes (ImmobilonP) by wet electrophoretic transfer. Blots were blocked for 1 h at RT with 5% non-fat dry milk in TBS/0.05% Tween20 and incubated overnight at 4° C. with the following primary antibodies: 1:300 anti-CD9 ab92726 (Abcam); 1:300 anti-TSG101 ab83 (Abcam); 1:1000 anti-EGFR 4267S (CST); 1:300 anti-CD63 sc-365604, (Santa Cruz); 1:200 anti-CD81 cs-166029 (Santa Cruz); 1:1000 anti-pEGFR Tyr1068 3777S (CST); 1:2000 anti-GRB2 610111 (BD Biosciences); 1:1000 anti-Shc 06-203 (Millipore); 1:5000 anti-GFP ab13970 (Abcam); 1:1000 GAPDH ab9483 (Abcam); 1:10,000 HRPconjugated β-actin a3854 (Sigma); 1:400 anti-RNA Pol II cat #39097 (Active Motif); 1:500 anti-Hsp90 ab1429 (Abcam); 1:500 anti-eIF3A ab86146 (Abcam); 1:500 anti-eIF4A1 ab31217 (Abcam). HRP-conjugated secondary antibodies (Sigma, 1:2000) were incubated for 1 h at room temperature. Washes after antibody incubations were done on an orbital shaker, four times at 10 min intervals, with 1×TBS 0.05% Tween20. Blots were developed with chemiluminescent reagents from Pierce.

Immunoprecipitation. Exosomes and cell protein extracts gently rocked at 4° C. for 2 h. The lysates were centrifuged at 14,000 g in a pre-cooled centrifuge for 15 minutes and the pellet was discarded. Protein A or G agarose/sepharose beads were washed twice with PBS and restored to a 50% slurry with PBS. A bead/slurry mix (100 μl) was added to 100 μg of exosomes protein extracts or 20 μg of cells protein extracts and incubated at 4° C. for 10 min. Beads were removed by centrifugation at 14,000 g at 4° C. for 10 minutes and pellets discarded. 10 μg of anti-eIF4A 1 antibody was added to 100 μL of exosomal lysate and incubated overnight at 4° C. on an orbital shaker. 100 μL of Protein A or G agarose/sepharose bead slurry were added and left at 4° C. overnight. After centrifugation the supernatant was discarded and beads washed 3 times with ice-cold Urea/SDS buffer. The agarose/sepharose beads were boiled for 5 minutes to dissociate the immunocomplexes from the beads. The beads were collected by centrifugation and immunoblot was performed on the supernatant.

Identification of amino acids using UPLC-MS. Exosomes were mixed with 200 μL of methanol spiked with the Internal Standard tryptophan-d5 and incubated for an hour at −20° C. After centrifugation at 16,000 g for 15 min at 4° C., 190 μL of the supernatants was collected and the solvent removed. Dried extracts were reconstituted in 15 μL of methanol, of which 10 μL were transferred to microtubes and derivatized. Chromatographic separation and mass spectrometric detection conditions employed are summarized in Table 1. The mass range, 50-1000 m/z, was calibrated with cluster ions of sodium formate. An appropriate test mixture of standard compounds was analyzed before and after the entire set of randomized duplicated sample injections, in order to examine the retention time stability and sensitivity of the LC/MS system throughout the course of the run.

TABLE 1
Chromatographic conditions for the amino acids platform.

| | |
|---|---|
| System | SQD |
| Column type | UPLC BEH C18, 1.0 × 100 mm, 1.7 um |
| Flow rate | 0.14 ml/min |
| Solvent A | H2O + 10 mM Ammonium Bicarbonate |
| | (+NH4OH until pH: 8.8) |
| Solvent B | CAN |
| (% B), time | 2%, 0 min |
| (% B), time | 8%, 6.5 min |
| (% B), time | 20%, 10 min |
| (% B), time | 30%, 11 min |
| (% B), time | 99.9%, 12 min |
| (% B), time | 2%, 14 min |
| Column temperature | 40° C. |
| Injection volume | 1 μl |
| Ionisation | ES+ |
| Source temperature | 120° C. |
| Nebulisation N2 flow | 600 l/hour |
| Nebulisation N2 temperature | 350° C. |
| Cone N2 flow | 10 l/hour |
| Capillary voltage | 3.2 kV |
| Cone voltage | 30 V |

Data were processed using the TargetLynx application manager for MassLynx 4.1 software (Waters Corp., Milford, USA). A set of predefined retention time, mass-to-charge ratio pairs, Rt-m/z, corresponding to metabolites included in the analysis are fed into the program. Associated extracted ion chromatograms (mass tolerance window=0.05 Da) are then peak-detected and noise-reduced in both the LC and MS domains such that only true metabolite related features are processed by the software. A list of chromatographic peak areas is then generated for each sample injection, using the Rt-m/z data pairs (retention time tolerance=6 s) as identifiers. Normalization factors were calculated for each metabolite by dividing their intensities in each sample by the recorded intensity of the internal standard in that same sample.

Digital qPCR. Digital RNA reaction was performed using 3 ng of cDNA, TaqMan® Universal Master mix and QuantStudio™ 3D Digital PCR Master Mix v1 (Applied Biosystems) according to manufacture recommendations. Using QuantStudio™ 3D Digital PCR Chip Loader (Applied Biosystems) a total of 14.5 μL of the mix were loaded to a QuantStudio™ 3D Digital PCR 20K Chip Kit vi (Applied Biosystems). PCR reaction was performed on GeneAmp® 9700 (Applied Biosystems) following manufacture protocol. The chips were imaged using QuantStudio™ 3D Digital PCR Instrument (Applied Biosystems).

TABLE 2
Digital PCR Primers.

| | | SEQ ID |
|---|---|---|
| Name | Sequence | NO |
| Met | AGTAGGTAGCGCG | 1 |
| | TCAGTCTCATAAT | |
| | CTGAAGGTCGTGA | |
| | GTTCGATCCTCAC | |
| | ACGGGGCA | |
| Leu | AGGCGCTGGATTA | 2 |
| | AGGCTCCAGTCTC | |
| | TTCGGAGGCGTGG | |
| | GTTCGAATCCCAC | |
| | CGCTGCCA | |
| Val | AGTGGTTATCACG | 3 |
| | TTCGCCTAACACG | |
| | CGAAAGGTCCCCG | |
| | GTTCGAAACCGGG | |
| | CGGAAACA | |
| Ser | GGCGATGGACTAG | 4 |
| | AAATCCATTGGGG | |
| | TTTCCCCGCGCAG | |
| | GTTCGAATCCTGC | |
| | CGACTACG | |

[³⁵S]methionine labeling of exosomes. Exosomes were isolated as previously described and resuspended in methionine-free culture medium without FBS with 0.1-1.0 mCi/ml trans label [³⁵S]-L-methionine (Amersham Biosciences) and incubated overnight. Alternatively exosomes were incubated in the presence of cycloheximide (Sigma, 100 μg/mL). Exosomes were pelleted, washed in ice-cold PBS and resuspended in Urea/SDS lysis buffer as previously described. Protein extracts were quantified using a BCA protein assay kit, run on acrylamide gels and transferred onto PVDF membranes (ImmobilonP) by wet electrophoretic transfer, after which the membranes were analyzed by autoradiography using the EN3HANCE® autoradiography enhancer according to the manufacturer's instructions (Perkin-Elmer).

Real-time PCR Analysis. DNase treated RNA was retro-transcribed with MultiScribe Reverse Transcriptase (Applied Biosystems) and oligo-d(T) primers following total exosomes RNA purification with Trizol (Invitrogen). Real-time PCR was performed on an ABI PRISM 7300HT Sequence Detection System Instrument using SYBR Green Master Mix (Applied Biosystems) and β-actin as the control. 28S rRNA primer pairs (QF00318857) and 18S rRNA primer pairs (QF00530467) were purchased as ready specific primer pairs from Qiagen. Other primers are listed below. Each measurement was performed in triplicate. Threshold cycle (Rothstein et al.), the fractional cycle number at which the amount of amplified target reached a fixed threshold, was determined and expression was measured using the 2-ΔCt formula, as previously reported (Livak and Schmittgen, 2001).

TABLE 3
qPCR Probes.

| | | SEQ |
|---|---|---|
| | | ID |
| Name | Sequence | NO |
| p21 F | 5′TACCCTTGTG | 5 |
| | CCTCGCTCAG3′ | |
| p21 R | 5′GAGAAGATC | 6 |
| | AGCCGGCGTTT3′ | |
| hsa-Actin F | 5′CATGTACGTT | 7 |
| | GCTATCCAGGC3′ | |
| hsa-Actin R | 5′CTCCTTAATG | 8 |
| | TCACGCACGAT3′ | |
| mmu-Actin F | 5′GGCTGTATTC | 9 |
| | CCCTCCATCG3′ | |
| mmu-Actin R | 5′CCAGTTGGT | 10 |
| | AACAATGCCATGT3′ | |

Lysate preparation for in vitro transcription and translation. Exosomes and cell pellets were washed once in ice-cold PBS and resuspended in an equal volume of ice-cold 20 mM HEPES (pH 7.5), 100 mM potassium acetate, 1 mM magnesium acetate, 2 mM dithiothreitol, and 100 μg/mL lysolecithin. After 1 min on ice, they were again pelleted and resuspended in an equal volume of ice-cold hypotonic extraction buffer. After 5 min on ice, the lysates were disrupted by passing 10 times through a 26-gauge needle attached to a 1-mL syringe. The resulting homogenates were centrifuged at 1000 g for 5 min at 4° C. The supernatant was collected, and aliquots were frozen in liquid nitrogen and stored at −80° C. for use in the in vitro translation assay.

In vitro coupled transcription and translation. Lysates obtained from cells and exosomes as previously described were used for in vitro translation in reaction volumes of 12 μL. Standard reaction conditions were as follows: cell lysate (final protein concentration 10 μg) or exosomes lysate (final concentration 100 μg), 1 μg pEMT7-GFP cDNA expression plasmid, 20 mM HEPES-KOH (pH 7.6), 80 mM potassium acetate, 1 mM magnesium acetate, 1 mM ATP, 0.12 mM GTP, 17 mM creatine phosphate, 0.1 mg/mL creatine phosphokinase, 2 mM dithiothreitol, 40 μM of each of the 20 amino acids, 0.15 mM spermidine, and 400 U/mL RNAsin (Promega). Incubations were carried out for 3 h at 37° C.

Electroporation and culture of exosomes. Exosomes were pelleted and resuspended in 400 μL of electroporation buffer (1.15 mM potassium phosphate pH 7.2, 25 mM potassium chloride, 21% Optiprep), with 20 μg of plasmid (pCMV-GFP, pEGFP-p53 Addgene plasmid 12091, pcDNA3-RLUC-POLIRES-FLUC and pcDNA-FLUC). Exosomes were electroporated using a 4 mm cuvette using a Gene Pulser Xcell Electroporation System (BioRad), as previously described (Alvarez-Erviti et al., 2011). When appropriate, exosomes were electroporated in the presence of cycloheximide (Sigma, 100 μg/mL) or α-amanitin (Sigma, 30 μg/mL), for inhibition of translation and transcription, respectively. Electroporated exosomes were cultured in serum-free DMEM at 37° C. for the time points indicated.

Flow cytometry analysis of electroporated exosomes. Exosomes preparations (5-10 μg) were incubated with 5 μL of 4 μm diameter aldehyde/sulfate latex beads (Interfacial Dynamics, Portland, Oreg.) and resuspended into 600 μL. Exosomes-coated beads were analyzed on a FACS Calibur flow cytometer (BD Biosciences) and analyzed for green fluorescence.

Exosomes delivery and confocal microscopy. MCF10A cells were plated at an appropriate confluency in 12-well plates on inserted coverslips and cultured overnight. The following day cells were incubated with MDA-MB-231 exosomes resuspended in serum-free culture DMEM for 2 h, washed with cold PBS 1× and fixed for 20 min at room temperature with 4% PFA/PBS. Slides were permeabilized for 10 min at RT with PBS 0.5% Triton X-100 and counterstained with DAPI. Images were obtained using a Zeiss LSM510 Upright Confocal System using the recycle tool to maintain identical settings. For data analysis, images were selected from a pool drawn from at least two independent experiments. Figures show representative fields.

Reverse transwell assay. Exosomes were isolated from MDA-MB-231 cells as previously described, and resuspended in PBS and quantified using Nanosight NTA. 10×10⁹ exosomes in 150 μL PBS were added to each bottom well of a 96-well Corning™ HTSTranswell® system. PBS alone was added to bottom wells as a negative control. An insert containing a polycarbonate membrane with 40 nm pores was added to each well, and 100 μL of PBS alone, PBS with 20% FBS, or PBS with 10,000 ng/ml of EGF were added to the insert. Trasnswell plates were incubated at 37° C. with 5% CO₂, and at samples were collected from the upper inserts after 4 h and 24 h incubation for exosomes quantification using Nanosight NTA.

Statistics. Error bars indicate±s.d. between biological replicates. Technical as well as biological triplicates of each experiment were performed. Statistical significance was calculated by Student's t-test, ANOVA or Mann-Whitney test, as appropriate and specified in the description of the figures.

### Example 1—EGFR Phosphorylation is Detected in Exosomes Derived from MDA-MB-231, Triple Negative Human Breast Cancer Cells

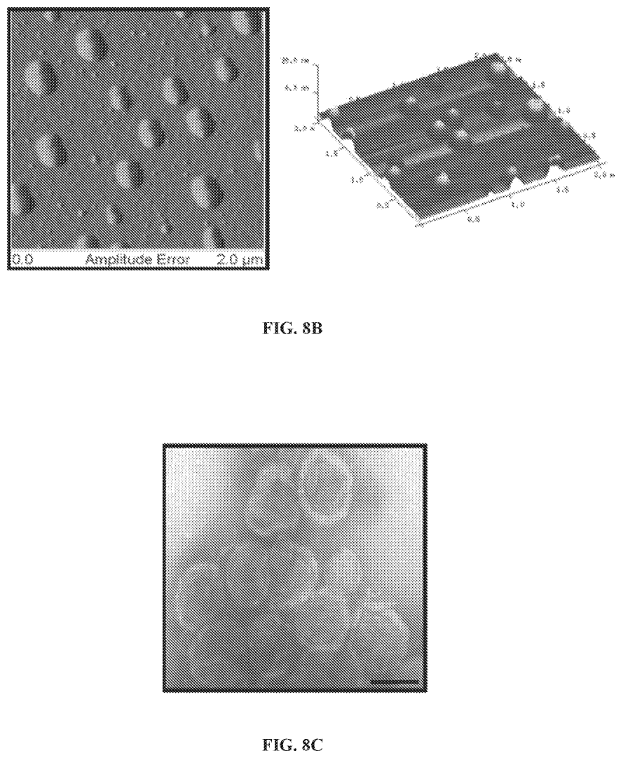

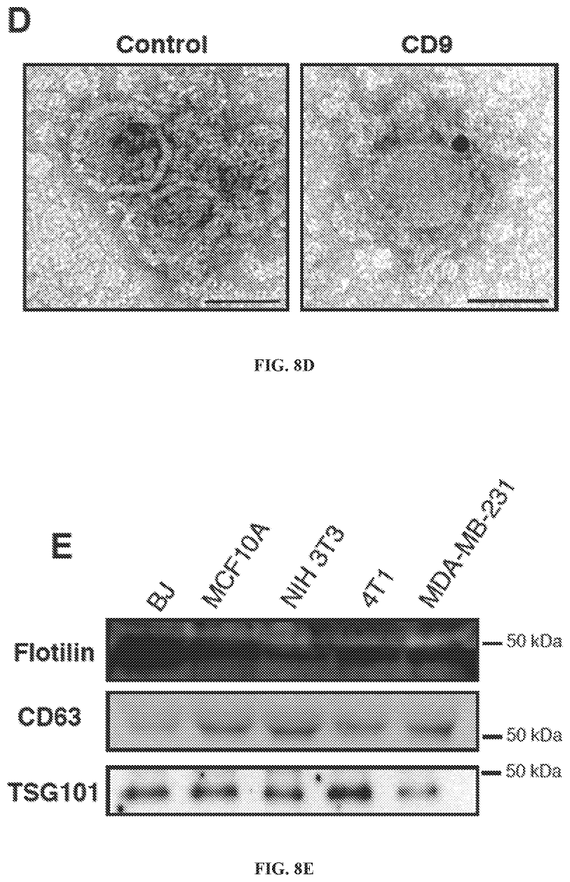

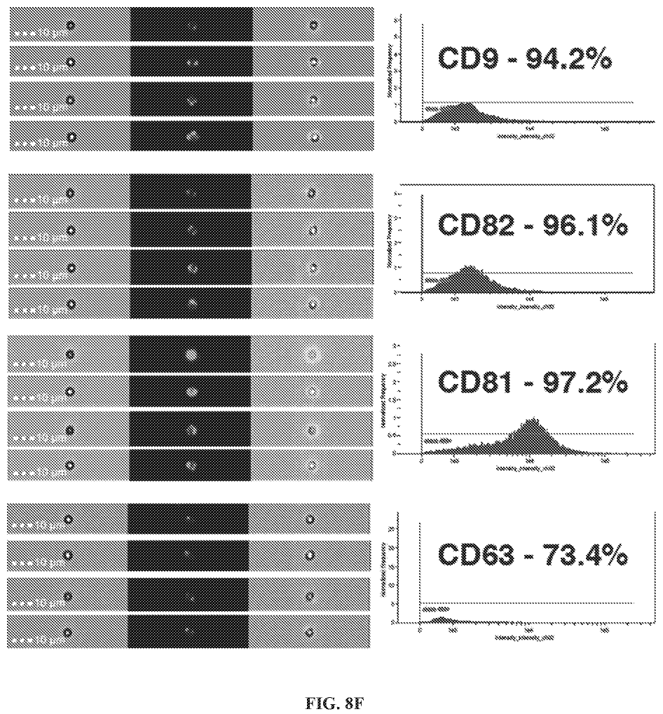

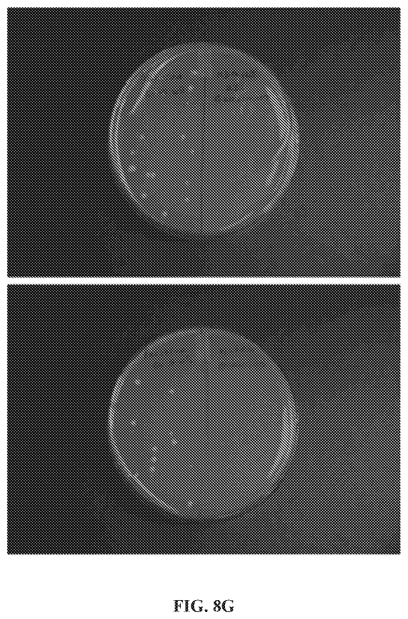

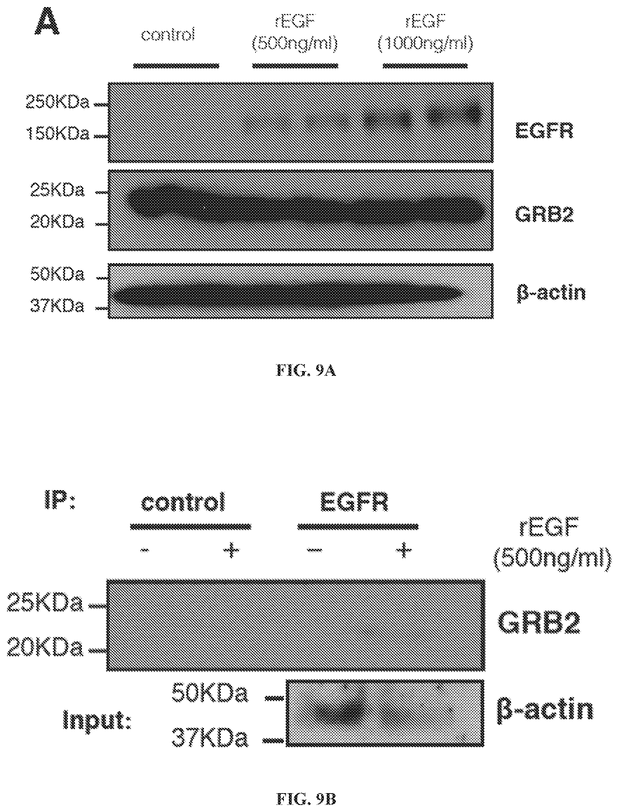

Exosomes were isolated from different murine and human cell lines using established ultracentrifugation techniques (Melo et al., 2015; Melo et al., 2014). The isolated exosomes represent a heterogeneous mix, with the same size distribution being consistently observed between preparations. NanoSight nanoparticle tracking analysis (NTA) as well as atomic force microscopy (AFM) revealed particles with a size distribution averaging 104 f 1.5 nm in diameter, and ranging roughly between 30 and 200 nm. This was confirmed by transmission electron microscopy (TEM) showing extracellular vesicles surrounded by a lipid bilayer (FIGS. 8A-C). The isolated exosomes were further shown by immunogold/TEM imaging, immunoblot analysis and imaging flow cytometry to possess known markers of exosomes (Raposo and Stoorvogel, 2013) (FIGS. 8D-F). To further confirm their purity, exosomes samples were inoculated onto solidified LB plates, showing no colony formation when compared to bacterial controls obtained from mouth swabs. This demonstrates the absence of bacterial contamination in the isolated exosomes (FIG. 8G).

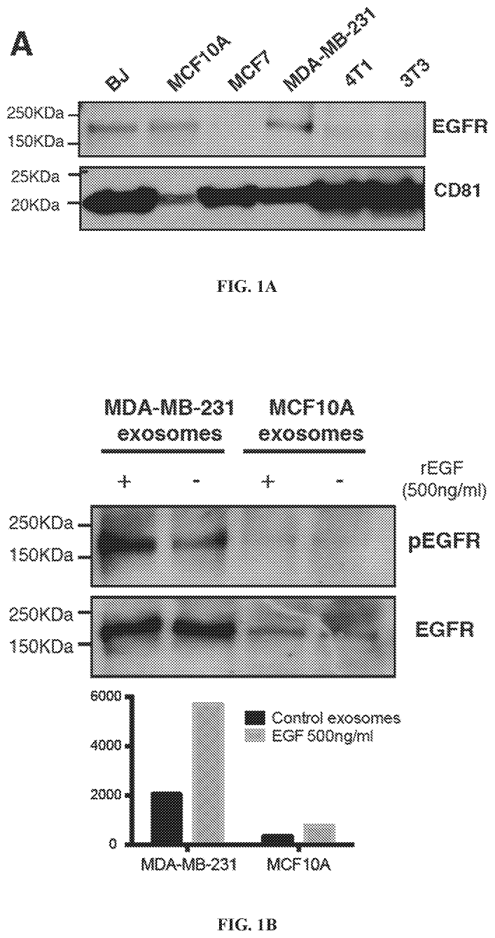

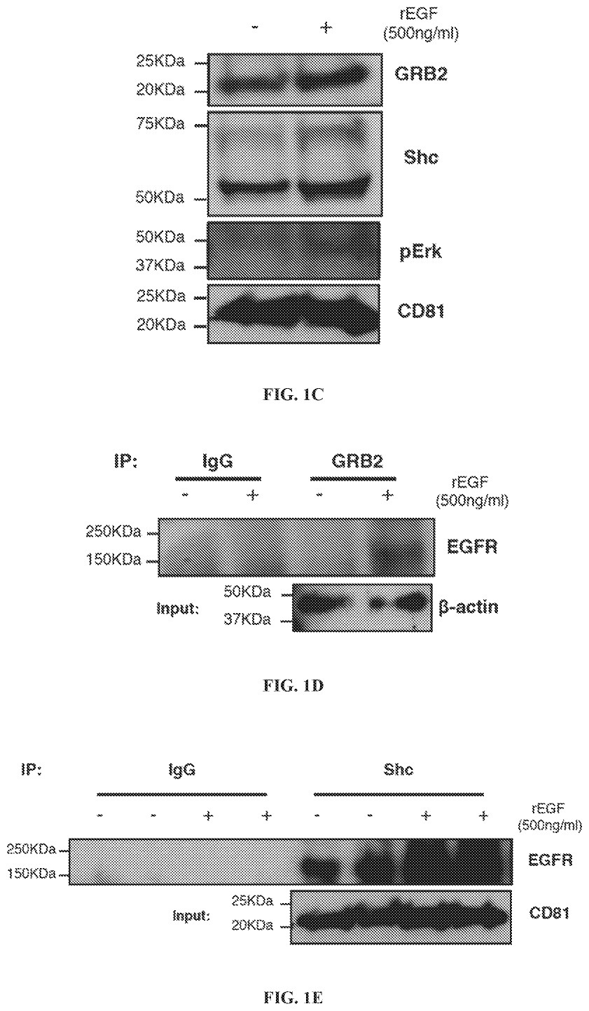

Exosomes obtained from different cell lines were probed by immunoblotting for their EGFR content. While exosomes from all cell lines show low levels of EGFR expression, exosomes derived from the BJ fibroblast cell line and MDA-MB-231 triple negative breast cancer cell line showed strong expression of the receptor. The known exosomes marker CD81 is shown as a loading control (FIG. 1A). Given the importance of EGFR for the progression of triple negative breast cancer (Lim et al., 2016; Liu et al., 2012; Nakai et al., 2016), its functional role in MDA-MB-231 derived exosomes was further explored. Exosomes were derived from MDA-MB-231 and MCF10A cells, and 1 billion exosomes were incubated with 500 ng/ml of recombinant human EGF (rhEGF) for 15 minutes at 37° C. in serum-free culture media. Immunoblotting of protein extracts obtained from these exosomes with an antibody specific for the Tyr1068 residue of EGFR revealed an increase in the phosphorylation levels of this receptor in exosomes derived from MDA-MB-231, but not non-tumorigenic MCF10A breast epithelial cells (FIG. 1B). Baseline levels of EGFR did not change in any of the samples, confirming the specificity of the observed increase in phosphorylation. Recombinant human EGF (rhEGF) stimulation also lead to an increase in the levels of phosphorylated ERK, suggesting that the observed EGFR phosphorylation triggers downstream signaling events within the exosomes (FIG. 1C). Further probing of the protein content of MDA-MB-231 exosomes showed that they also contained downstream effectors of EGFR, namely GRB2 and Shc (FIG. 1C).

It was then investigated whether upon rhEGF stimulation, exosomal EGFR could engage its downstream adaptors. Exosomes were stimulated with rhEGF for 15 minutes at 37° C. The exosomal protein extracts were subjected to pull down assays using specific antibodies for GRB2 and Shc, and it was detected that upon EGF stimulation they showed increased co-immunoprecipitation with EGFR (FIGS. 1D,E). Isotype IgGs were used as a negative control for the pull down, and did not reveal EGFR co-immunoprecipitation. Additionally, by reversing the assay and pulling down EGFR, it was also possible to detect coimmunoprecipitated GRB2 only in EGF stimulated exosomes (FIG. 8B). Taken together these results demonstrate that exosomes from MDA-MB-231 cells contain EGFR that can be phosphorylated by incubation with its ligand in cell-free conditions, leading to putative downstream signaling events within the exosomes.

### Example 2—EGF Stimulation of Exosomes Alters their Protein Content

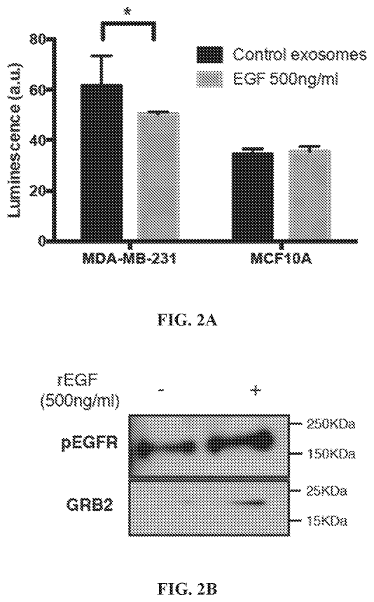

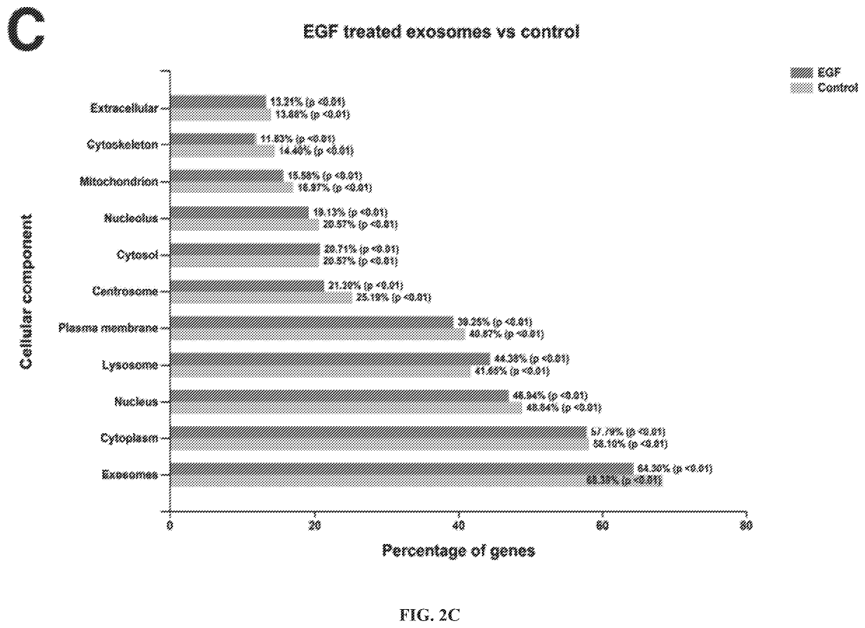

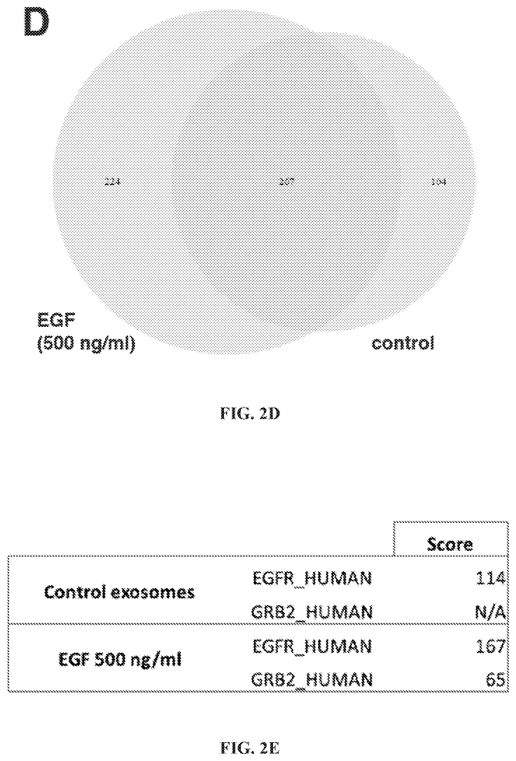

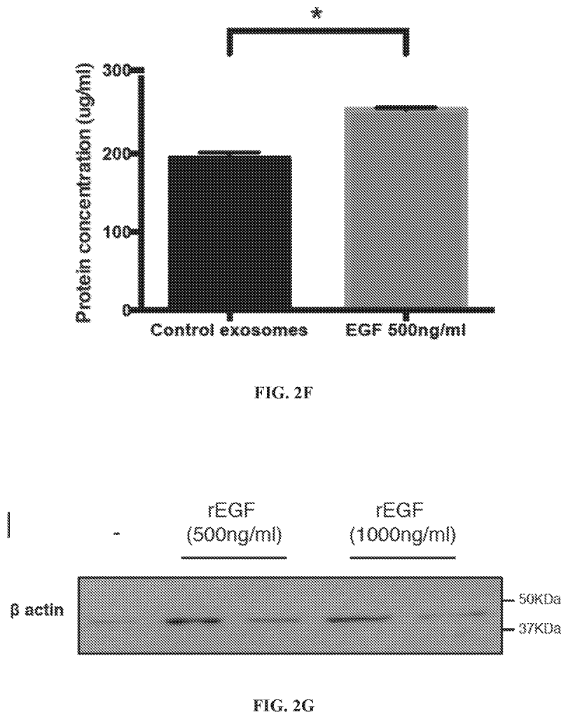

Receptor tyrosine kinases require ATP as a substrate for their kinase activity, and prostate-derived exosomes have been shown to have the capacity to generate ATP (Ronquist et al., 2013a). To further confirm the existence of phosphorylation activity in the absence of cells, an ATP quantification assay was performed on exosomes with or without rhEGF stimulation. Using a luminescence based kit, ATP was detected in exosomes from both MDA-MB-231 cells and MCF10A cells, albeit in smaller quantities in the latter. Exosomes from MDA-MB-231 cells, but not MCF10A cells, demonstrated a slight decrease in their ATP quantity upon stimulation with EGF (FIG. 2A). To further investigate the impact of EGF stimulation on exosomes, they were incubated them in cell-free conditions for a period of 48 h. The levels of GRB2 protein levels were consistently higher in exosomes stimulated with EGF for 48 h, compared to their unstimulated counterparts (FIG. 2B). This raised the intriguing possibility that the protein content of exosomes might have changed upon growth factor stimulation. To further investigate this possibility, mass spectrometry analysis was performed on protein extracts obtained from rhEGF unstimulated or stimulated exosomes. Protein extracts were subjected to trypsin digestion and evaluated using an ESI-TRAP mass spectrometer to obtain an MS/MS peptide spectrum for each sample. The obtained spectra were then evaluated against a SwissProt database for peptide identification to obtain a list of proteins for each exosomes sample. Using the open access FunRich functional enrichment analysis tool (Pathan et al., 2015) it was observed that the majority of identified hits in both the unstimulated and stimulated exosomes matched proteins previously identified in exosomes (Vesiclepedia database) (FIG. 2C). A higher number of proteins were identified in exosomes stimulated with rhEGF when compared to the unstimulated ones (491 vs. 371, FIG. 2D). While the majority of these proteins were common to both stimulated and unstimulated exosomes, 224 out of 491 proteins were detected only upon rhEGF stimulation. While EGFR was identified on both samples, GRB2 was only identified in rhEGF stimulated exosomes (FIG. 2E). It should be stressed however that this does not mean that GRB2 is not present in unstimulated exosomes, but it might be present at a level under the detectable threshold for this type of analysis. The Exponentially Modified Protein Abundance Index (emPAI), which allows for label-free quantification of relative changes in protein content based on the observable peptide matches, was employed (Ishihama et al., 2005). The top 15 proteins that revealed a stronger increase in rhEGF stimulated exosomes when compared to their unstimulated counterparts included several participants of actin remodeling and membrane dynamics, such as a-actinin, MARCKS, ezrin, moesin, and integrin alpha-2 (Tables 4&5). A gene ontology (GO) analysis was then performed using the PANTHER overrepresentation test. Interestingly, among the top GO biological processes enriched in the rhEGF stimulated exosomes, several were related to actin remodeling and migration (5 out of the 20 top pathways, Table 6).

TABLE 4
Top 15 proteins identified as upregulated in exosomes from MDA-MB-231 cells incubated
with 500 ng/ml of EGF at 37° C. for 48 h compared to control exosomes,
based on the protein scores using the emPAI method (Ishihama et al., 2005).

| | | | EGF- | Fold |
|---|---|---|---|---|
| Protein Name | Description | Control | Treatment | Change |
| CHMP2A | Charged multivesicular body | 0.4 | 2.25 | 5.625 |
| | protein 2a OS = Homo sapiens | | | |
| | GN = CHMP2A PE = 1 SV = 1 | | | |
| CHMP2B | Charged multivesicular body | 0.19 | 1.03 | 5.421052632 |
| | protein 2b OS = Homo sapiens | | | |
| | GN = CHMP2B PE = 1 SV = 1 | | | |
| ACSL4 | Long-chain-fatty-acid--CoA ligase | 0.06 | 0.31 | 5.166666667 |
| | 4 OS = Homo sapiens | | | |
| | GN = ACSL4 PE = 1 SV = 2 | | | |
| LDHB | L-lactate dehydrogenase B chain | 0.12 | 0.59 | 4.916666667 |
| | OS = Homo sapiens GN = LDHB | | | |
| | PE = 1 SV = 2 | | | |
| ACTN1 | Alpha-actinin-1 OS = Homo sapiens | 0.04 | 0.18 | 4.5 |
| | GN = ACTN1 PE = 1 SV = 2 | | | |
| ITGA2 | Integrin alpha-2 OS = Homo sapiens | 0.07 | 0.26 | 3.714285714 |
| | GN = ITGA2 PE = 1 SV = 1 | | | |
| HIST2H2AA3 | Histone H2A type 2-A | 6.9 | 24.85 | 3.601449275 |
| | OS = Homo sapiens | | | |
| | GN = HIST2H2AA3 PE = 1 SV = 3 | | | |
| MARCKS | Myristoylated alanine-rich C-kinase | 0.14 | 0.5 | 3.571428571 |
| | substrate OS = Homo sapiens | | | |
| | GN = MARCKS PE = 1 SV = 4 | | | |
| VPS37B | Vacuolar protein sorting- | 0.14 | 0.5 | 3.571428571 |
| | associated protein 37B | | | |
| | OS = Homo sapiens | | | |
| | GN = VPS37B PE = 1 SV = 1 | | | |
| RPL5 | 60S ribosomal protein L5 | 0.13 | 0.45 | 3.461538462 |
| | OS = Homo sapiens | | | |
| | GN = RPL5 PE = 1 SV = 3 | | | |
| EHD2 | EH domain-containing protein 2 | 0.07 | 0.23 | 3.285714286 |
| | OS = Homo sapiens | | | |
| | GN = EHD2 PE = 1 SV2 | | | |
| DNAJA1 | DnaJ homolog subfamily A | 0.61 | 1.84 | 3.016393443 |
| | member 1 OS = Homo sapiens | | | |
| | GN = DNAJA1 PE = 1 SV = 2 | | | |
| EZR | Ezrin OS = Homo sapiens | 1.23 | 3.39 | 2.756097561 |
| | GN = EZR PE = 1 SV = 4 | | | |
| MSN | Moesin OS = Homo sapiens | 1.74 | 4.48 | 2.574712644 |
| | GN = MSN PE = 1 SV = 3 | | | |
| ARF1 | ADP-ribosylation factor 1 | 0.5 | 1.26 | 2.52 |
| | OS = Homo sapiens | | | |
| | GN = ARF1 PE = 1 SV = 2 | | | |

TABLE 5
Top 15 proteins identified as downregdated in exosomes from
MDA-MB-231 cells incubated with 500 ng/ml of EGF at 37°
C. for 48 h compared to control exosomes, based on the protein
scores using the emPAI method (Ishihania et al., 2005).

| | | | EGF- | Fold |
|---|---|---|---|---|
| Protein Name | Description | Control | Treatment | Change |
| KRT9 | “Keratin, type 1 cytoskeletal 9 | 6.86 | 1.28 | 0.186588921 |
| | OS = Homo sapiens | | | |
| | GN = KRT9 PE = 1 SV = 3” | | | |
| KRT2 | “Keratin, type II cytoskeletal 2 | 2.46 | 0.92 | 0.37398374 |
| | epidermal OS = Homo sapiens | | | |
| | GN = KRE2 PE = 1 SV = 2” | | | |
| GOLGA7 | Golgin subfamily A member 7 | 0.7 | 0.3 | 0.428571429 |
| | OS = Homo sapiens | | | |
| | GN = GOLGA7 PE = 1 SV = 2 | | | |
| CALM1 | Calmodulin OS = Homo sapiens | 0.64 | 0.28 | 0.4375 |
| | GN = CALM1 PE = 1 SV = 2 | | | |
| HRAS | GTPase HRas OS = Homo sapiens | 0.48 | 0.22 | 0.458333333 |
| | GN = HRAS PE = 1 SV = 1 | | | |
| SCAMP2 | Secretory carrier-associated | 0.26 | 0.12 | 0.461538462 |
| | membrane protein 2 | | | |
| | OS = Homo sapiens | | | |
| | GN = SCAMP2 PE = 1 SV = 2 | | | |
| RGS19 | Regulator of G-protein signaling 19 | 0.41 | 0.19 | 0.463414634 |
| | OS = Homo sapiens | | | |
| | GN = RGS19 PE = 1 SV = 1 | | | |
| RAB5B | Ras-related protein Rab-5B | 0.43 | 0.2 | 0.465116279 |
| | OS = Homo sapiens | | | |
| | GN = RAB5B PE = 1 SV = 1 | | | |
| CORO1C | Coronin-1C OS = Homo sapiens | 0.17 | 0.08 | 0.470588235 |
| | GN = CORO1C PE = 1 SV = 1 | | | |
| SLC7A11 | Cystine/glutamate transporter | 0.17 | 0.08 | 0.470588235 |
| | OS = Homo sapiens | | | |
| | GN = SLC7A11 PE = 1 SV = 1 | | | |
| PACSIN3 | Protein kinase C and casein kinase | 0.19 | 0.09 | 0.473684211 |
| | substrate in neurons protein 3 | | | |
| | OS = Homo sapiens | | | |
| | GN = PACSIN3 PE = 1 SV = 2 | | | |
| VPS4B | Vacuolar protein sorting- | 0.19 | 0.09 | 0.473684211 |
| | associated protein 4B | | | |
| | OS = Homo sapiens | | | |
| | GN = VPS4B PE = 1 SV = 2 | | | |
| OR51E1 | Olfactory receptor 51E1 | 0.27 | 0.13 | 0.481481481 |
| | OS = Homo sapiens | | | |
| | GN = OR51E1 PE = 2 SV = 1 | | | |

TABLE 6
Top 20 gene ontology (GO) pathways identified based on the differential protein scores
between control exosomes and exosomes incubated with 500 ng/ml EGF at 37° C.
for 48 h. A list of differentially expressed proteins was obtained using the emPAI
method and used as input for GO analysis using the PANTHER overrepresentation test.

| GO | Homo | | | | | |
|---|---|---|---|---|---|---|
| biological | sapiens - | | | | upload_1 | |
| process | REFLIST | upload_1 | upload_1 | upload_1 | (fold | upload_1 |
| complete | (21002) | (113) | (expected) | (over/under) | Enrichment) | (P-value) |
| actomyosin | 3 | 3 | 0.02 | + | >100 | 5.77E−03 |
| contractile | | | | | | |
| ring | | | | | | |
| organization | | | | | | |
| (GO: 0044837) | | | | | | |
| actomyosin | 3 | 3 | 0.02 | + | >100 | 5.77E−03 |
| contractile | | | | | | |
| ring assembly | | | | | | |
| (GO: 0000915) | | | | | | |
| assembly of | 3 | 3 | 0.02 | + | >100 | 5.77E−03 |
| actomyosin | | | | | | |
| apparatus | | | | | | |
| involved in | | | | | | |
| cytokinesis | | | | | | |
| (GO: 0000912) | | | | | | |
| positive | 4 | 3 | 0.02 | + | >100 | 1.36E−03 |
| regulation of | | | | | | |
| extracellular | | | | | | |
| exosome | | | | | | |
| assembly | | | | | | |
| (GO: 1903553) | | | | | | |
| regulation of | 6 | 4 | 0.03 | + | >100 | 3.58E−04 |
| extracellular | | | | | | |
| exosome | | | | | | |
| assembly | | | | | | |
| (GO: 1903551) | | | | | | |
| viral release | 6 | 3 | 0.03 | + | 92.93 | 4.56E−02 |
| from host cell | | | | | | |
| (GO: 0019076) | | | | | | |
| movement in | 6 | 3 | 0.03 | + | 92.93 | 4.56E−02 |
| environment | | | | | | |
| of other | | | | | | |
| organism | | | | | | |
| involved in | | | | | | |
| symbiotic | | | | | | |
| interaction | | | | | | |
| (GO: 0052192) | | | | | | |
| movement in | 6 | 3 | 0.03 | + | 92.93 | 4.56E−02 |
| host | | | | | | |
| environment | | | | | | |
| (GO: 0052126) | | | | | | |
| exit from host | 6 | 3 | 0.03 | + | 92.93 | 4.56E−02 |
| cell | | | | | | |
| (GO: 0035891) | | | | | | |
| exit from host | 6 | 3 | 0.03 | + | 92.93 | 4.56E−02 |
| (GO: 0035890) | | | | | | |
| cell separation | 17 | 7 | 0.09 | + | 76.53 | 6.98E−08 |
| after | | | | | | |
| cytokinesis | | | | | | |
| (GO: 0000920) | | | | | | |
| ESCRT III | 10 | 4 | 0.05 | + | 74.34 | 2.72E−03 |
| complex | | | | | | |
| disassembly | | | | | | |
| (GO: 1904903) | | | | | | |
| ESCRT | 10 | 4 | 0.05 | + | 74.34 | 2.72E−03 |
| complex | | | | | | |
| disassembly | | | | | | |
| (GO: 1904896) | | | | | | |
| positive | 15 | 6 | 0.08 | + | 74.34 | 2.69E−06 |
| regulation of | | | | | | |
| exosomal | | | | | | |
| secretion | | | | | | |
| (GO: 1903543) | | | | | | |
| regulation of | 16 | 6 | 0.09 | + | 69.7 | 3.94E−06 |
| exosomal | | | | | | |
| secretion | | | | | | |
| (GO: 1903541) | | | | | | |
| regulation of | 15 | 5 | 0.08 | + | 61.95 | 2.09E−04 |
| mitotic | | | | | | |
| spindle | | | | | | |
| assembly | | | | | | |
| (GO: 1901673) | | | | | | |
| positive | 16 | 5 | 0.09 | + | 58.08 | 2.88E−04 |
| regulation of | | | | | | |
| viral release | | | | | | |
| from host cell | | | | | | |
| (GO: 1902188) | | | | | | |
| viral budding | 23 | 7 | 0.12 | + | 56.57 | 5.64E−07 |
| (GO: 0046755) | | | | | | |
| viral budding | 20 | 6 | 0.11 | + | 55.76 | 1.48E−05 |
| via host | | | | | | |
| ESCRT | | | | | | |
| complex | | | | | | |
| (GO: 0039702) | | | | | | |

Taken together, these mass spectrometry data suggest that MDA-MB-231 exosomes can alter their protein content upon rhEGF stimulation. These data also suggest that the same exosomes stimulated with rhEGF could undergo actomyosin remodeling and migration, indicative of a motility phenotype in response to rhEGF stimulation. A bicinchoninic acid (BCA) assay for protein quantification confirmed the increase in protein content in exosomes stimulated with rhEGF, when compared with unstimulated controls (FIG. 2F). Immunoblotting for R-actin also shows an increase in the levels of polymerized actin in exosomes stimulated with different amounts of rhEGF when compared with control unstimulated exosomes (FIG. 2G). Collectively, these observations indicate an unexpected degree of biological activity in exosomes. Therefore, the possibility that exosomes are capable of synthesizing proteins de novo under permissive conditions was further investigated and the potential induction of exosomes motility upon growth factor stimulation was explored.

### Example 3—Exosomes Derived from Different Cell Types Contain the Functional Constituents Required for Transcription and Translation

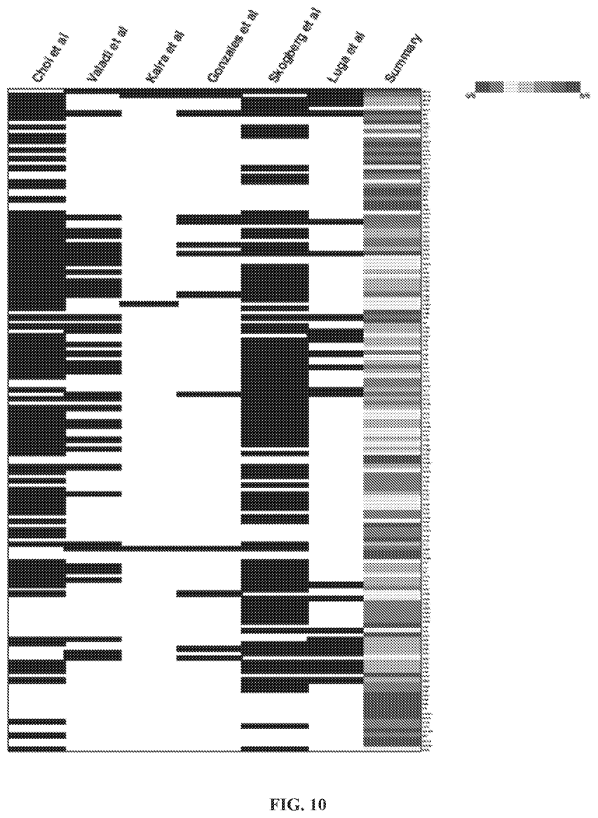

Analysis of proteomics data from exosomes of different cellular origins revealed the presence of several constituents of the protein synthesis machinery, such as eukaryotic initiation factors, ADP ribosylation factors, and ribosomal proteins (Choi et al., 2012; Melo et al., 2015; Pisitkun et al., 2004; Valadi et al., 2007) (FIGS. 10, 11A,B). This information, taken together with the knowledge that mRNAs and their corresponding proteins are found in exosomes, further suggested that isolated exosomes could possess the capacity to translate mRNA into proteins.

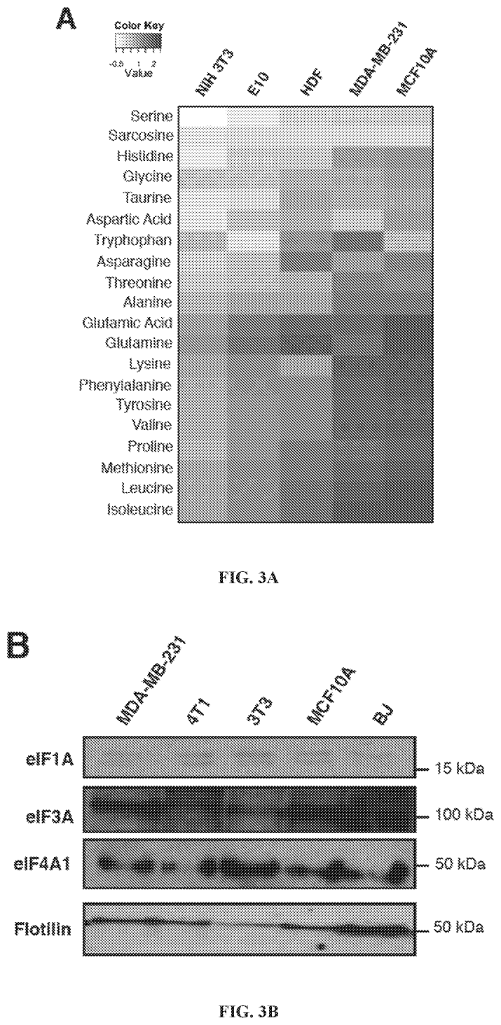

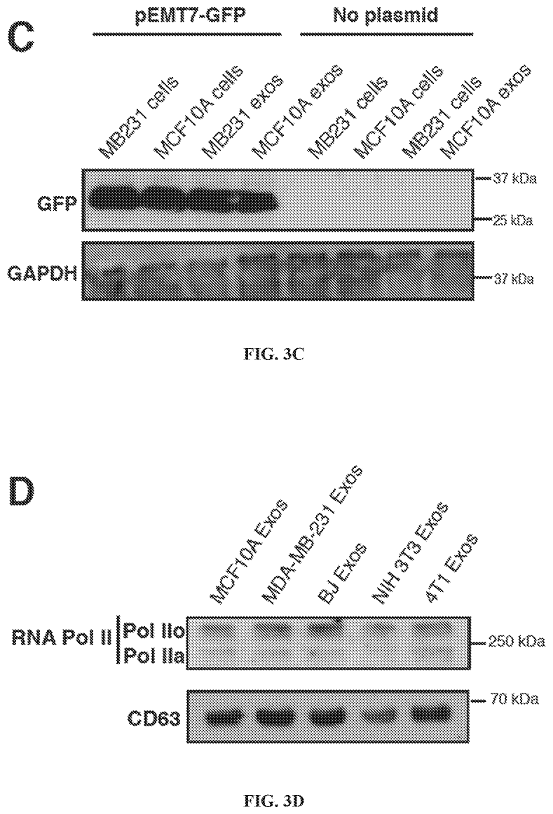

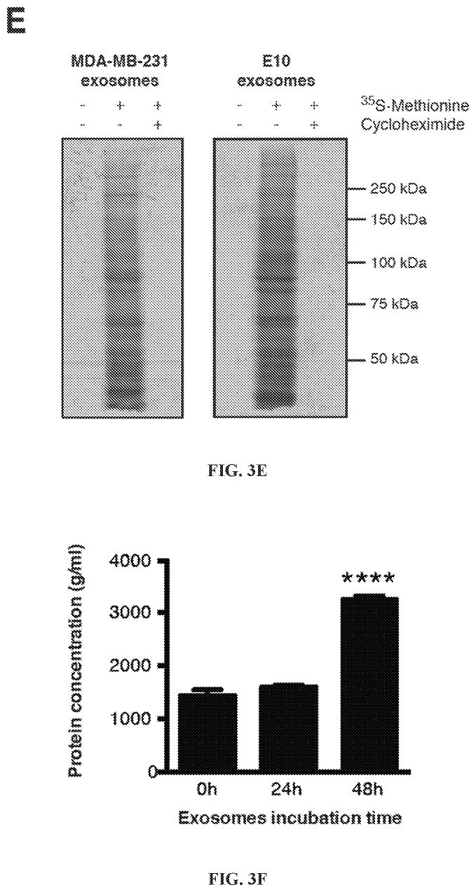

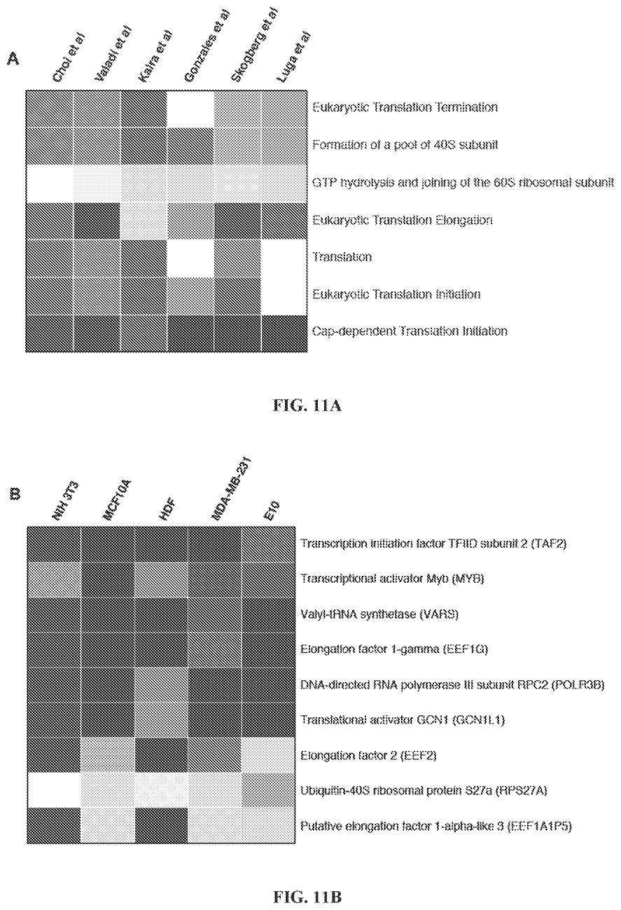

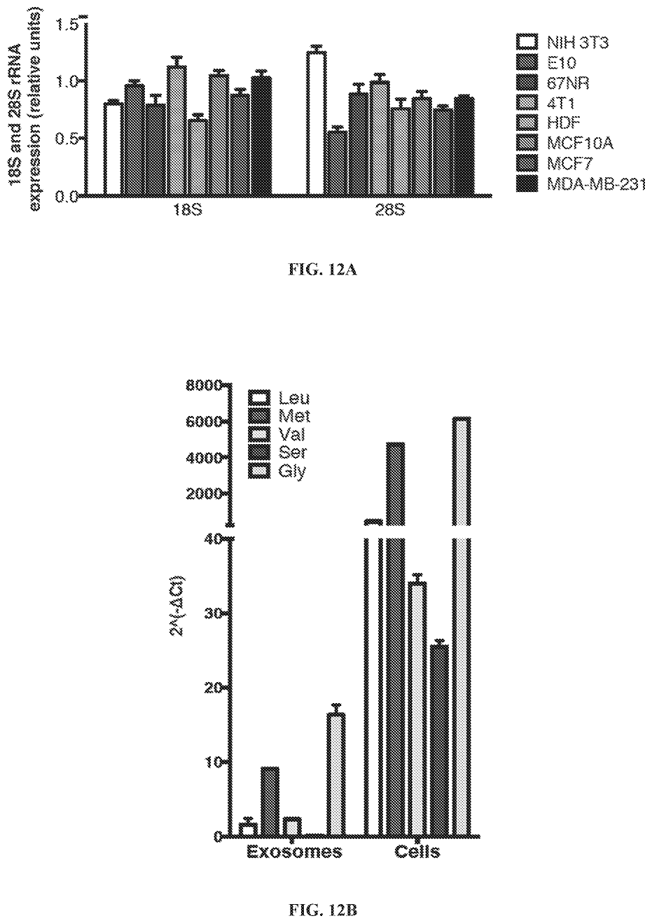

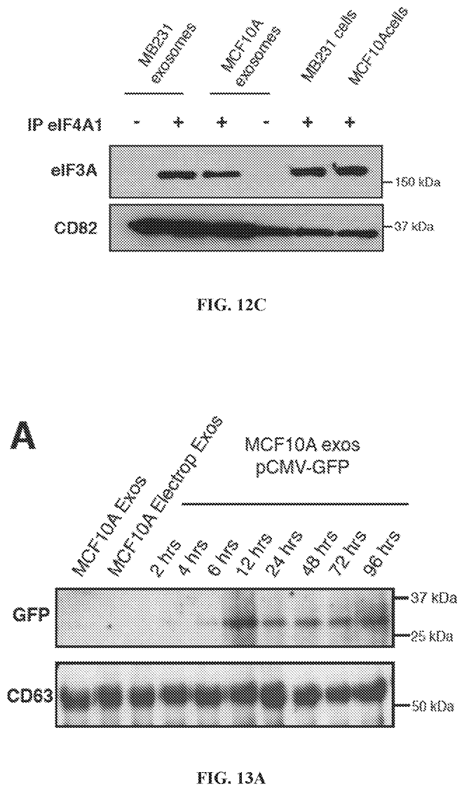

Using quantitative PCR (qPCR) analysis, the presence of both 18S and 28S rRNAs was confirmed, as well as tRNAs for methionine, glycine, leucine, serine, and valine in all analyzed exosomes (FIGS. 12A,B). Additionally, Ultra Performance Liquid Chromatography-Mass Spectrometry (UPLC-MS) analysis of exosomes revealed the existence of all free amino acids (FIG. 3A). Immunoblotting analysis identified the presence of different members of the translation initiation complex in exosomes, including eIF4A, eIF3A, and eIF1A (FIG. 3B), confirming the observations made through mass spectrometry analysis. In addition, initiation factors eIF4A and eIF3A co-immunoprecipitate in protein extracts obtained from exosomes (Morino et al., 2000) (FIG. 12C).

To functionally address the relevance of constituents for protein production present in exosomes, total protein extracts of exosomes isolated from MCF10A and MDA-MB-231 cells were incubated with a cDNA expression plasmid for green fluorescent protein (GFP plasmid) and a coupled in vitro transcription and translation assay was performed. Western Blot analysis of the extracts after incubation with the GFP encoding plasmid revealed production of GFP protein (FIG. 3C). The fact that exosomes lysates from both MDA-MB-231 and MCF10A cells allowed for the synthesis of protein from the GFP expression plasmid confirmed that exosomes derived from different cells likely contain all the necessary functional components for both DNA transcription and mRNA translation. Consistent with the potential for DNA transcription, additional immunoblot analysis of protein extracts from exosomes isolated from different cell sources identified the presence of RNA polymerase II subunits, both in its phosphorylated and non-phosphorylated forms (FIG. 3D).

### Example 4—Exosomes are Capable of Cell-Independent Protein Synthesis

To further validate the finding that exosomes have the autonomous capability for de novo mRNA translation, isolated exosomes obtained from MDAMB-231 cells as well as the murine lung cancer E10 cell line were incubated with ³⁵S-methionine to enable labeling of newly synthesized proteins by the exosomes. The assay was performed at 37° C. in order to activate the putative biosynthetic processes and potential autocrine stimulation. Autoradiography of protein extracts from exosomes incubated for 72 h in the presence of ³⁵S-methionine exhibited the incorporation of the radioactive amino acid into several proteins in the range of 40 to 300 kDa. This was largely inhibited when exosomes were incubated with the protein translation inhibitor cycloheximide along with ³⁵S-methionine (FIG. 3E). A distinct pattern of labeled proteins was observed when exosomes derived from different cancer cells were incubated with ³⁵S-methionine. Additionally, total protein content was quantified from freshly isolated exosomes incubated in cell-free culture media. After 48 h of incubation, the total exosomal protein content was significantly increased (FIG. 3F).

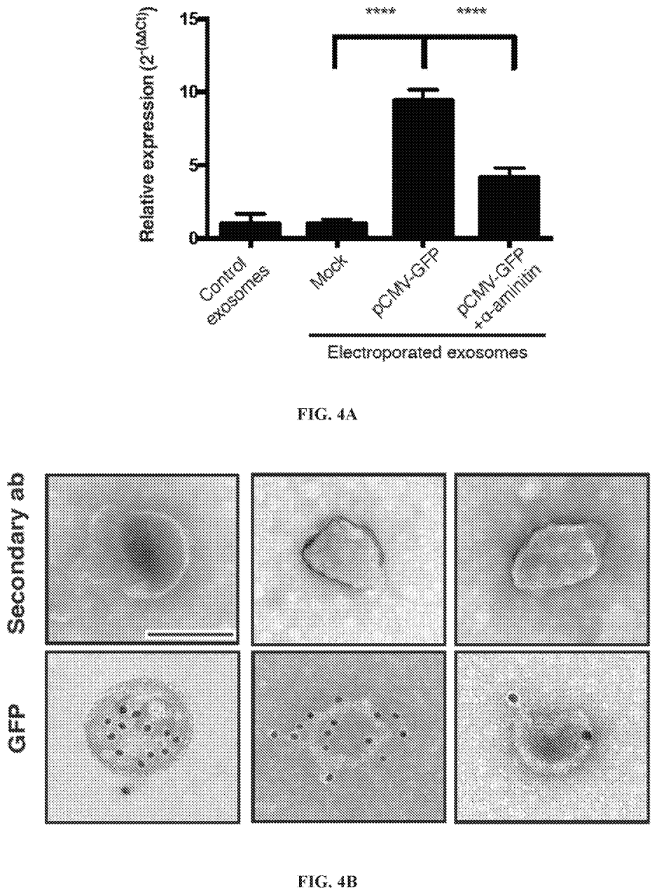

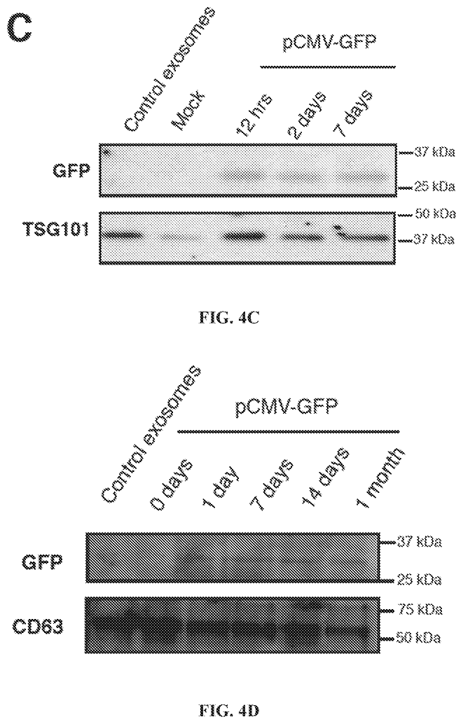

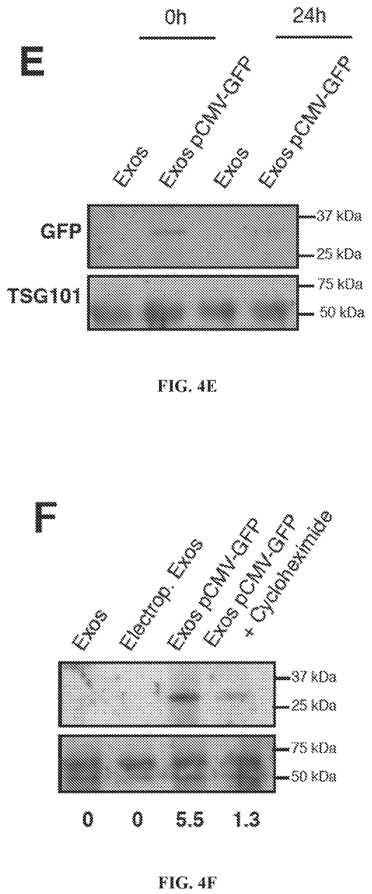

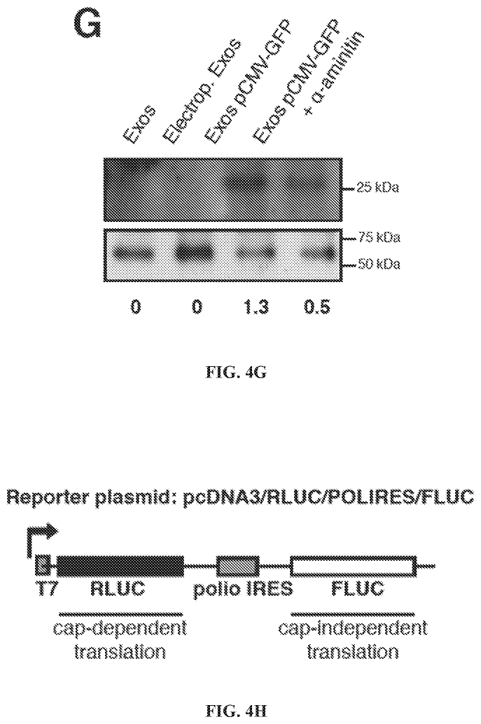

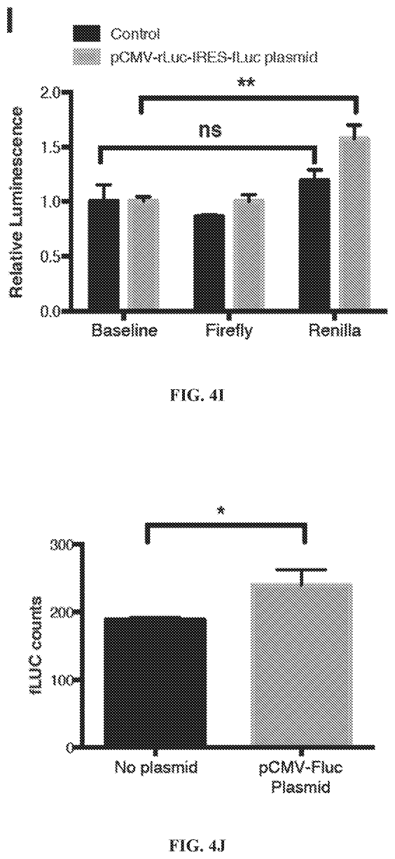

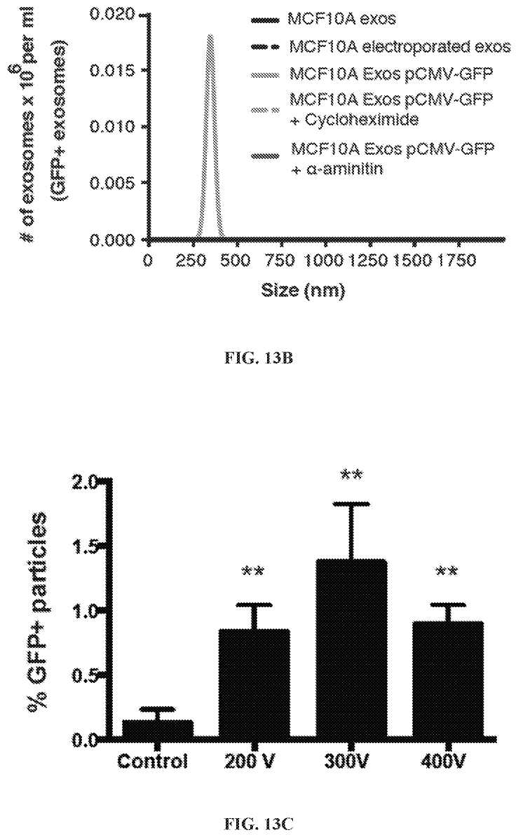

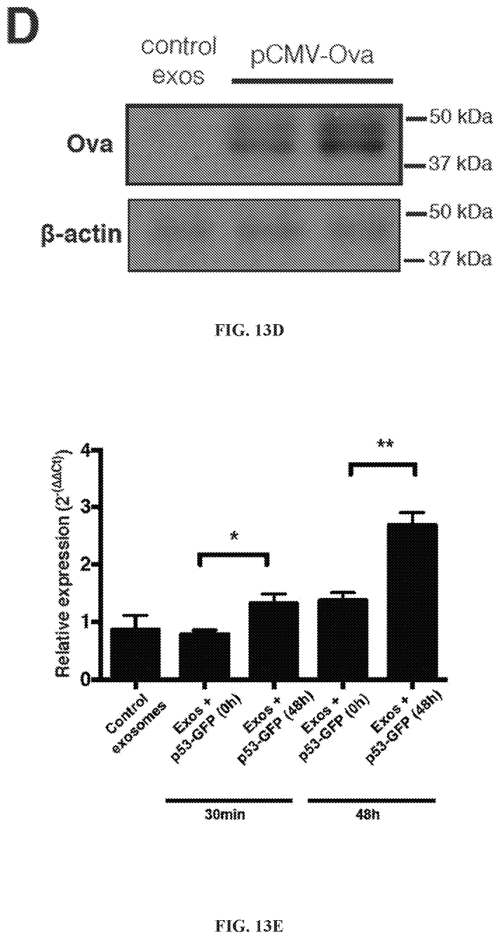

Next, whether transcription and translation can take place in intact exosomes, rather than just their lysates, was confirmed by setting up a protocol of exosomes in vitro translation. A pCMV-GFP expression plasmid was electroporated directly into exosomes derived from MDA-MB-231 cells (Borges et al., 2013; El-Andaloussi et al., 2012; Kamerkar et al., 2017) and the electroporated exosomes were incubated at 37° C. in serum-free culture media, for 48 h. qPCR analysis of isolated exosomal RNA after digestion with DNAse revealed the presence of GFP mRNA in exosomes electroporated with the pCMV-GFP expression plasmid (FIG. 4A). Transmission electron microscopy showed that the structure of the exosomes electroporated with the pCMV-GFP plasmid was intact, and immunogold labeling using an anti-GFP antibody showed that the protein could only be detected in the GFP plasmid-containing exosomes (FIG. 4B). Immunoblot analysis of exosomes protein extracts using a GFP antibody further confirmed the presence of GFP in pCMV-GFP plasmid electroporated exosomes, observed as early as 12 h after electroporation (FIG. 4C). GFP could be observed in exosomes electroporated with the expression plasmid after 1 week and up to 1 month (FIGS. 4C,D), albeit without any increase over the levels observed at 24 h. The same pattern was also observed in exosomes derived from MCF10A cells, confirming that exosomes derived from different cells, not just tumorigenic, contain all the required constituents and have the capacity for de novo protein synthesis (FIG. 13A).

Immunoblot analysis of exosomes electroporated with a GFP plasmid showed a reduction of about 80% in GFP levels when incubated in the presence of the protein translation inhibitor cycloheximide (FIG. 4E). GFP production was also decreased in the presence of α-amanitin, a transcription inhibitor of RNA polymerase II (FIGS. 4E,F). NanoSight NTA of electroporated exosomes using a 488 nm laser also detected green fluorescence in exosomes electroporated with the pCMV-GFP plasmid but not in mock-electroporated exosomes or exosomes electroporated with the plasmid and cycloheximide or α-amanitin (FIG. 13B). Additionally, beads based flow cytometry analysis of plasmid-containing exosomes using different electroporation conditions detected the presence of GFP (FIG. 13C). Next, exosomes were incubated for 24 h at 37° C. to initiate biological processes, before electroporation with pCMV-GFP plasmid. The GFP production, as detected by immunoblotting, was impaired, suggesting an exhaustion of the required components for transcription and translation in the exosomes that are pre-incubated at 37° C. (FIG. 4G).

To confirm that these results were not specific to just GFP, an ovalbumin expression plasmid (pCMV-Ova), a protein that is also not expressed in mammalian cells, was used. As with GFP, immunoblotting analysis of exosomes after electroporation and incubation at 37° C. for 48 h showed production of ovalbumin only in exosomes electroporated with the pCMV-Ova plasmid (FIG. 13D).

Initiation of protein translation of most mRNAs in eukaryotes involves recognition of the 5′ cap structure by the eIF4F complex (Merrick, 2004). To determine whether protein translation in exosomes is cap-dependent, a cDNA bicistronic construct was employed consisting of two different luciferase cistrons separated by an internal ribosome entry site (FIG. 4H) (Poulin et al., 1998). In this system, translation of Renilla luciferase is cap-dependent, whereas translation of firefly luciferase is directed by the poliovirus IRES, and is therefore cap-independent (FIG. 4H). Electroporation of the plasmid directly into exosomes led to an increase in Renilla Luciferase activity with no apparent change in Firefly Luciferase activity (FIG. 4I) (Poulin et al., 1998), suggesting that protein translation in exosomes occurs in a cap-dependent manner. Since Firefly and Renilla Luciferase enzymes have different activity requirements, this assay was repeated using a plasmid where Firefly luciferase is expressed under the control of a CMV promoter. The luciferase activity was also observed in the pCMV-Fluc electroporated exosomes (FIG. 4J).

### Example 5—mRNA Translation in Exosomes Generates Functional Proteins and can be Stimulated by Growth Factors

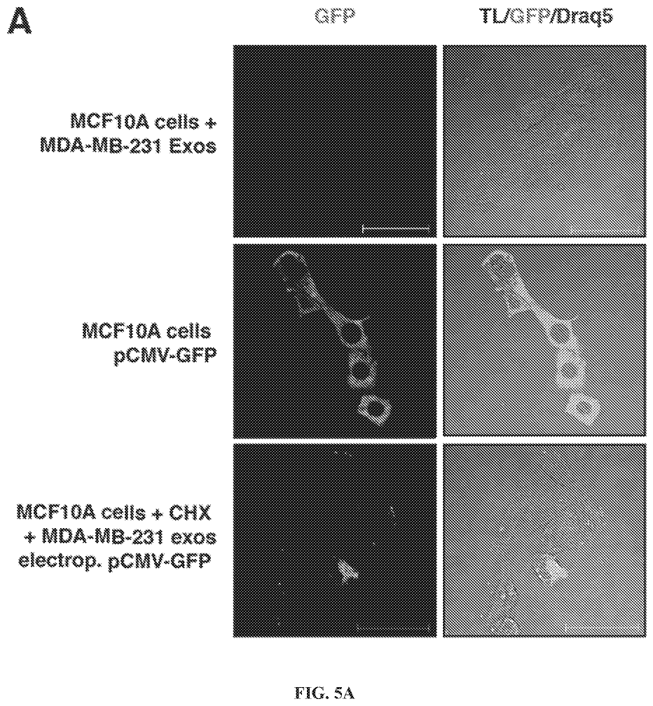

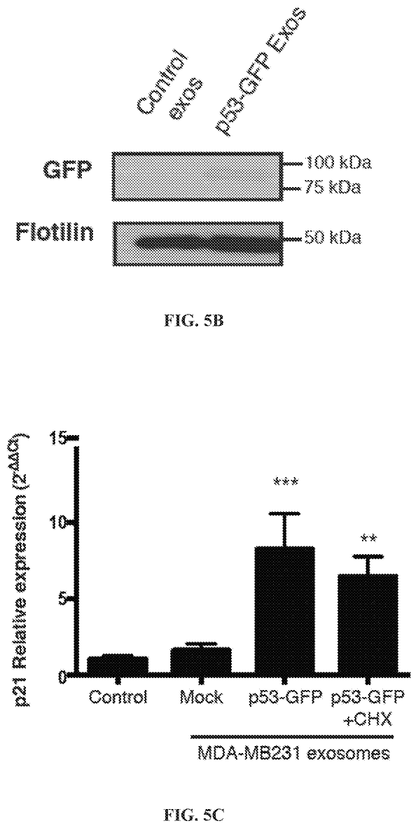

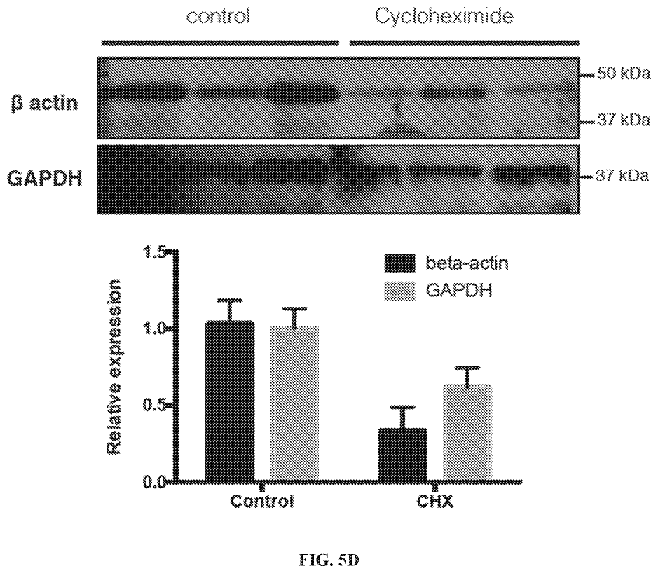

MCF10A cells pretreated with cycloheximide were incubated with MDA-MB-231 exosomes that were directly electroporated with the pCMV-GFP plasmid. Imaging by confocal microscopy detected green fluorescence in the MCF10A cells, likely contributed by the GFP protein (after transcription and translation) delivered by MDAMB-231 exosomes (FIG. 5A, top and bottom panels). Interestingly, cells directly electroporated with the pCMV-GFP plasmid show a GFP fluorescence pattern distinct from fluorescence pattern observed in cells incubated with pCMV-GFP plasmid containing exosomes (FIG. 5A, middle and bottom panels).

MDA-MB-231 cells overexpress an inactive mutant form of the tumor suppressor protein p53, which is therefore unable to activate the p21 promoter (Gartel et al., 2003). Wild-type (wt) p53 typically responds to DNA damage by direct induction of p21, facilitating cell cycle arrest (Zilfou and Lowe, 2009). Exosomes isolated from MDA-MB-231 cells were electroporated with a plasmid encoding for wt p53 fused to GFP. The electroporated exosomes were incubated in culture media for 48 h to allow transcription and translation to generate wt p53 protein (FIG. 5B). Subsequently, exosomes containing the newly formed wt p53 were incubated with recipient MDA-MB-231 cells under the influence of cycloheximide. The recipient MDA-MB-231 cells revealed a substantial increase in expression of p21 (FIG. 5C), confirming the functionality of wt p53 protein that was exclusively synthetized by the exosomes (FIG. 5B). To additionally confirm that this increase in p21 expression was indeed due to wt p53 protein newly translated by exosomes rather than due to delivery of the plasmid, MDA-MB-231 derived exosomes were electroporated with the p53-GFP plasmid and either allowed to incubate for 48 h to synthesize the wt p53 protein prior to incubation with the recipient MDA-MB-231 cells (48 h), or delivered immediately to the recipient MDA-MB-231 cells without allowing them to produce the wt p53 protein (0 h, FIG. 13E). Exosomes that were allowed to actively synthesize wt p53 protein 48 h prior to delivery induced p21 expression in the recipient MDA-MB-231 cells as early as 30 minutes post exosomes incubation, higher than when compared to exosomes with just the plasmid, which show the same baseline p21 expression observed in control MDA-MB-231 cells (FIG. 13E).

In order to further demonstrate that exosomes from MDA-MB-231 cells exhibit a baseline capacity for intrinsic protein synthesis, exosomes were incubated at 37° C., with and without the presence of cycloheximide. Immunoblotting of protein extracts from these exosomes showed a consistent reduction in the expression levels of small cytoplasmic proteins, β-actin and GAPDH, upon incubation with cycloheximide, again demonstrating the existence of a baseline level of protein synthesis in these exosomes (FIG. 5D).

In order to determine which proteins are produced by the MDA-MB-231 exosomes in the absence of external stimuli, an adapted version of stable isotope labeling with amino acids in culture (SILAC) was performed. Exosomes derived from MDA-MB-231 cells were incubated in SILAC medium supplemented with heavy labeled ¹³C-Lysine and ¹⁵N-Arginine. MDA-MB-231 exosomes were incubated in heavy labeled SILAC medium for 5 days and protein extracts were obtained, trypsin digested, and subjected to mass spectrometry analysis. While only a small number of heavy-labeled peptides matched the obtained MS/MS spectra, 11 proteins were able to be identified each matching 1 or 2 peptides containing the heavy-labeled amino acids (Table 7). This confirms that, albeit at low levels, baseline mRNA translation occurs in exosomes leading to the formation of very small amounts of newly synthesized proteins.

TABLE 7
List of proteins containing peptides matching spectra with heavy isotopes, obtained from mass spectrometry
analysis of protein extracts of MDA-MB-231 exosomes incubated with ¹³C-Lysine and ¹⁵N-Arginine SILAC
medium for 5 days. Each protein listed contains at least 1 peptide matching a ¹³C-Lysine
or ¹⁵N-Arginine heavy-labeled spectra.

| | | | | # | # Unique | # | # | # | MW | calc. |
|---|---|---|---|---|---|---|---|---|---|---|
| Acession | Description | Score | Coverage | Proteins | peptides | Peptides | PSMs | AAs | [kDa] | PI |
| Q7Z4S6 | Kinesin-like protein | 51.34 | 1.31% | 1 | 1 | 3 | 4 | 1674 | 187.1 | 6.42 |
| | KIF21A | | | | | | | | | |
| | OS = Homo sapiens | | | | | | | | | |
| | GN = KIF21A | | | | | | | | | |
| | PE = 1 SV = 2 - | | | | | | | | | |
| | [KI21A_HUMAN) | | | | | | | | | |
| O76038 | Secretagogin | 32.14 | 5.80% | 1 | 1 | 1 | 2 | 276 | 32 | 5.41 |
| | OS = Homo sapiens | | | | | | | | | |
| | GN = SCGN | | | | | | | | | |
| | PE = 1 SV = 2 - | | | | | | | | | |
| | [SEGN_HUMAN] | | | | | | | | | |
| Q9NQY0 | Bridging integrator 3 | 26.41 | 4.35% | 1 | 1 | 1 | 4 | 253 | 29.6 | 7.47 |
| | OS = Homo sapiens | | | | | | | | | |
| | GN = BIN3 | | | | | | | | | |
| | PE = 1 SV = 1 - | | | | | | | | | |
| | [BIN3_HUMAN] | | | | | | | | | |
| P05305 | Endothelin-1 | 57.22 | 8.96% | 1 | 2 | 3 | 3 | 212 | 24.4 | 9.41 |
| | OS = Homo sapiens | | | | | | | | | |
| | GN = EDN1 | | | | | | | | | |
| | PE = 1 SV = 1 - | | | | | | | | | |
| | [EDN1_HUMAN] | | | | | | | | | |
| P07476 | Involucrin | 40.81 | 1.71% | 1 | 1 | 1 | 2 | 585 | 68.4 | 4.61 |
| | OS = Homo sapiens | | | | | | | | | |
| | GN = IVL | | | | | | | | | |
| | PE = 1 SV = 2 - | | | | | | | | | |
| | [INVO_HUMAN] | | | | | | | | | |
| O15014 | Zinc finger protein 609 | 40.51 | 1.49% | 1 | 1 | 2 | 2 | 1411 | 151.1 | 8.03 |
| | OS = Human sapiens | | | | | | | | | |
| | GN = ZNF609 | | | | | | | | | |
| | PE = 1 SV = 2 - | | | | | | | | | |
| | [ZN609_HUMAN] | | | | | | | | | |
| Q9H0I3 | Coiled-coil domain- | 40.91 | 4.51% | 1 | 1 | 2 | 2 | 377 | 44.2 | 8.73 |
| | containing protein 113 | | | | | | | | | |
| | OS = Homo sapiens | | | | | | | | | |
| | GN = CCDC113 | | | | | | | | | |
| | PE = 2 SV = 1 - | | | | | | | | | |
| | [CC113_HUMAN] | | | | | | | | | |
| Q8IYE0 | Coiled-coil domain | 57.08 | 3.87% | 1 | 1 | 3 | 5 | 955 | 112.7 | 8.48 |
| | containing protein 146 | | | | | | | | | |
| | OS = Homo sapiens | | | | | | | | | |
| | GN = CCDC146 | | | | | | | | | |
| | PE = 2 SV = 2 - | | | | | | | | | |
| | [CC146_HUMAN] | | | | | | | | | |
| Q96KM6 | Zinc finger protein 512B | 23.93 | 1.23% | 1 | 1 | 1 | 1 | 892 | 97.2 | 9.83 |
| | OS = Homo sapiens | | | | | | | | | |
| | GN = ZNF512B | | | | | | | | | |
| | PE = 1 SV = 1 - | | | | | | | | | |
| | [Z512B_HUMAN] | | | | | | | | | |
| O15018 | PDZ domain- | 81.48 | 1.62% | 1 | 1 | 4 | 5 | 2839 | 301.5 | 7.43 |
| | containing protein 2 | | | | | | | | | |
| | OS = Homo sapiens | | | | | | | | | |
| | GN = PDZD2 | | | | | | | | | |
| | PE = 1 SV = 1 - | | | | | | | | | |
| | [PDZD2_HUMAN] | | | | | | | | | |
| P43308 | Translocon- | 24.02 | 3.83% | 1 | 1 | 1 | 1 | 183 | 20.1 | 8.35 |
| | associated protein | | | | | | | | | |
| | subunit beta | | | | | | | | | |
| | OS = Homo sapiens | | | | | | | | | |
| | GN = SSR2 | | | | | | | | | |
| | PE = 1 SV = 1 - | | | | | | | | | |
| | [SSRB_HUMAN] | | | | | | | | | |

Next, the protein translation assay was repeated using exosomes derived from MDA-MB-231 cells with electroporated pCMV-GFP plasmid. The exosomes were incubated in serum-free culture media at 37° C. for 48 h with or without stimulation with rhEGF. While the unstimulated exosomes presented with a baseline level of GFP production, the GFP levels increased upon incubation with different concentrations of rhEGF (FIG. 5E). This again confirmed that, while all exosomes can synthetize proteins, growth factor stimulation can alter their rate of production by leading to increased levels of protein synthesis.

### Example 6—Exosomes Actively Migrate in Response to Stimulation by rhEGF and Serum Factors

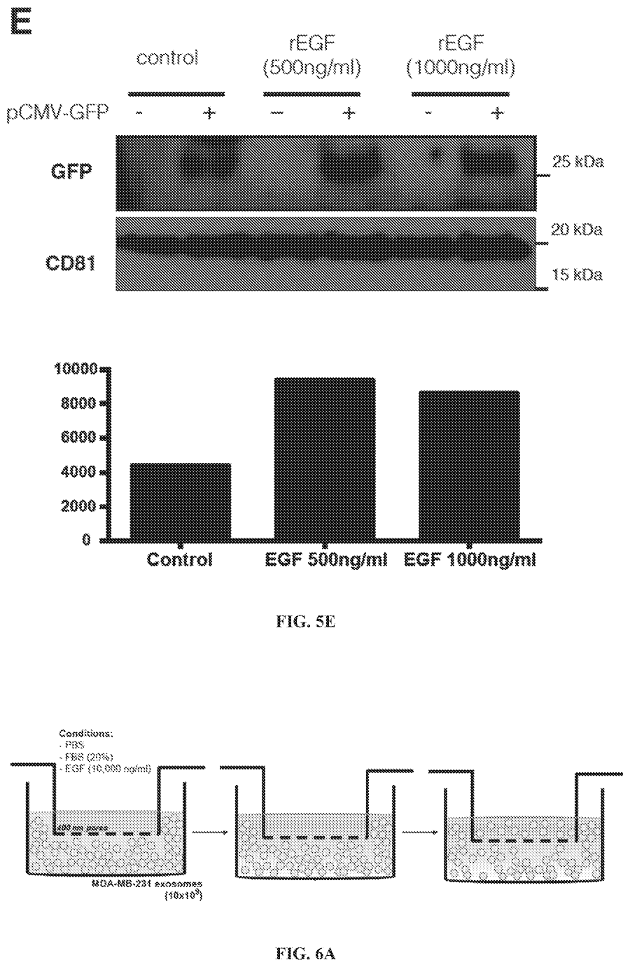

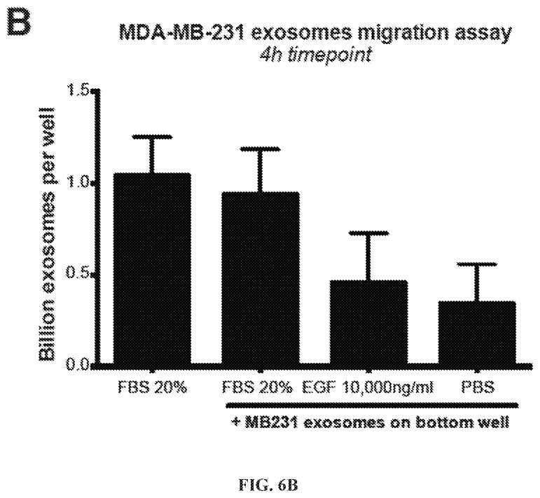

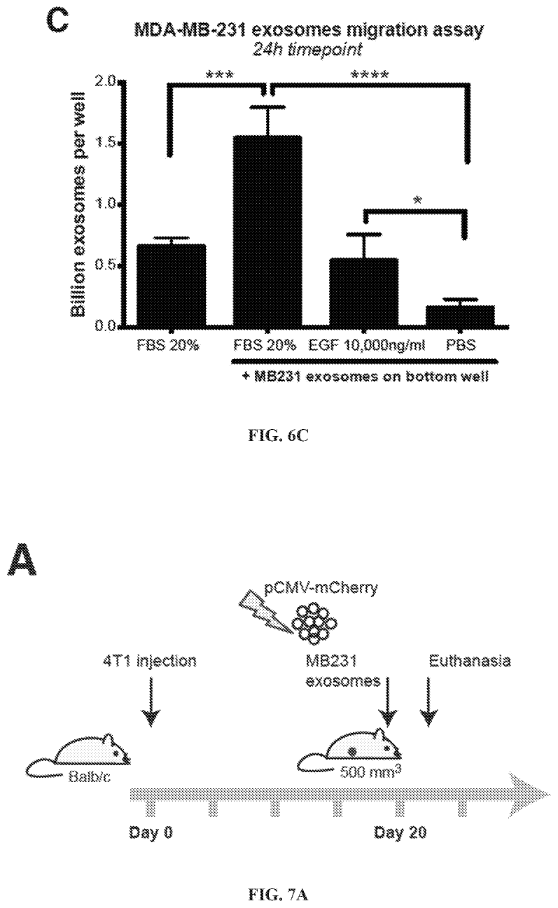

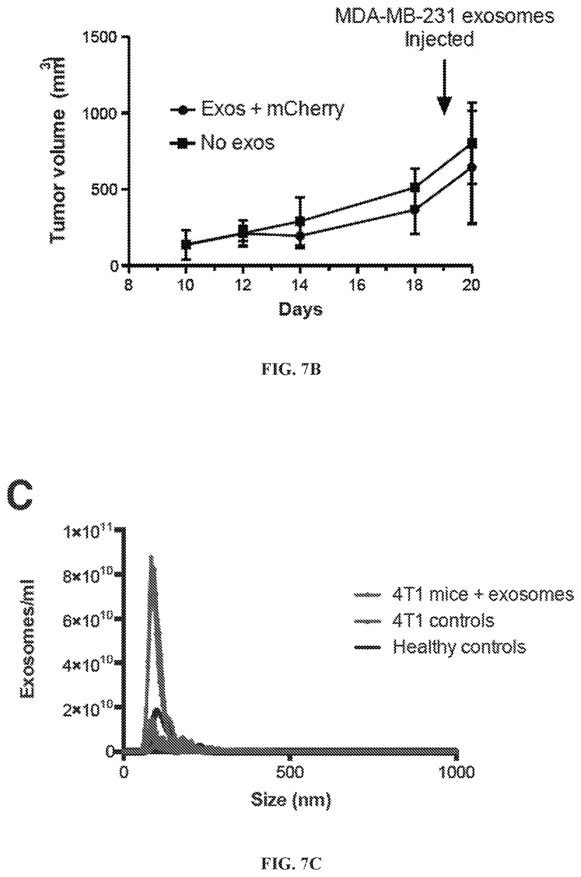

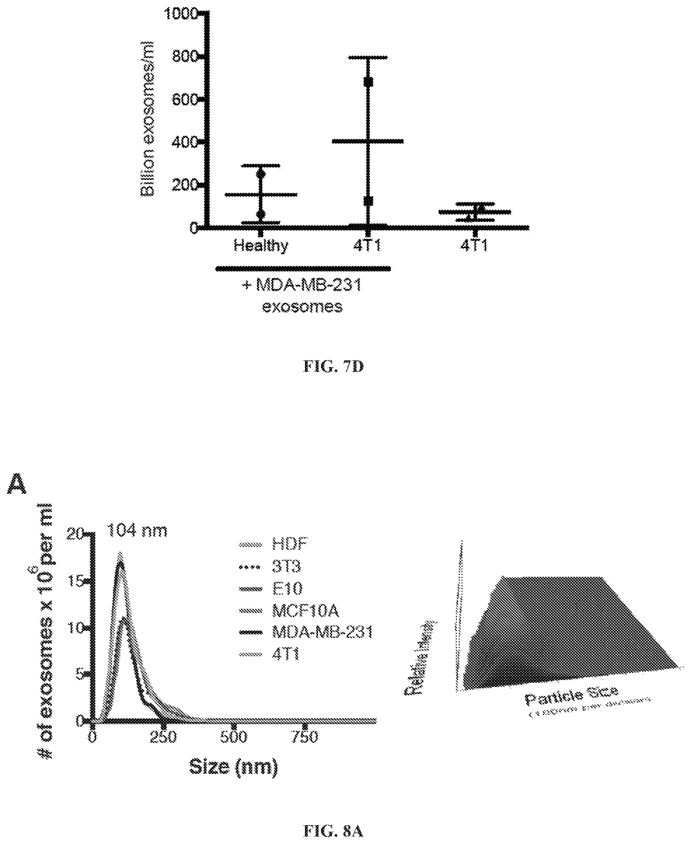

In order to determine whether growth factors can induce a motility phonotype in exosomes, a reverse migration assay based on the Boyden chamber system was designed including rhEGF and serum factors. Ten billion exosomes isolated from MDA-MB-231 cells were placed in the culture wells of a 96-well plate, covered by a polycarbonate surface insert containing 400 nm pores. The insert contained either PBS, PBS with 10 μM rhEGF, or PBS with 20% exosomes-depleted FBS (FIG. 6A). Because exosomes-depleted FBS could still contain trace amounts of exosomes (data not shown), 20% FBS was placed on the top insert with no exosomes in the bottom well, as a control. After incubation at 37° C., samples were obtained from the top insert at different time points and exosomes quantified using Nanosight NTA.

After a 4 h incubation, the levels of exosomes on the top insert were comparable across all experimental groups (FIG. 6B). After a 24 h incubation, 20% FBS significantly increased migration of the exosomes from the bottom to the top, suggesting a sustained chemotactic influence on MDA-MB-231 exosomes towards the higher serum growth factor gradient (FIG. 6C). The inserts with 20% FBS over wells with no exosomes had significantly fewer exosomes after 24 h, confirming the identity of the migrating exosomes as being from MDA-MB-231 cells (FIG. 6C). PBS resulted in negligible amounts of exosomes migration but rhEGF alone also induced motility of exosomes, albeit at a lower level when compared to complete serum associated growth factors (FIG. 6C). Taken together, these results suggest that exosomes exhibit functional chemotactic capacity that can be induced by growth factors.

### Example 7—Exosomes Specifically Exhibit Enhanced Protein Production in Tumor Bearing Mice

To address whether the capacity of exosomes to respond to the growth factor gradient induced by tumors involves intrinsic production of new proteins with functional consequences, a reference mouse model was generated. Mice with established 4T1 mammary tumors were injected with 5 billion MDA-MD 231/CD63-mCherry exosomes electroporated with either GFP or ovalbumin expression plasmids. Control experimental arms of this study included CD63-mCherry exosomes without the plasmids and CD63-mCherry exosomes electroporated with plasmid and cyclohexamide. Twenty-four hours after the I.P. injection of exosomes in tumor-bearing mice or non-tumor-bearing mice, the tumor, serum, and several other organs were collected. Exosomes were FACS isolated using the CD63 mCherry tag and evaluated for GFP or ovalbumin protein. GFP and ovalbumin were predominantly detected in the tumor, lung, bone, brain, and serum of mice with tumors, but were found only at very low levels in the tissues of non-tumor bearing mice and in tumor bearing mice that were injected with cyclohexamide-containing exosomes. These results demonstrate that while exosomes might be detected in the liver, lung, and brain of the non-tumor bearing mice, the exosomes enter these organs and more robustly (presumably via enhanced motility) including the tumor tissue, and biologically respond by generating de novo proteins. Additionally, the serum-derived exosomes from tumor bearing mice exhibit protein production, suggesting that tumors biologically influence exosomes at a systemic level.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,870,287
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,846,945
Aakalu et al., Dynamic visualization of local protein synthesis in hippocampal neurons. Neuron, 30:489-502, 2001.
Almoguera et al., Most human carcinomas of the exocrine pancreas contain mutant c-K-ras genes. Cell, 53:549-554, 1988.
Al-Nedawi et al., Endothelial expression of autocrine VEGF upon the uptake of tumor-derived microvesicles containing oncogenic EGFR. Proc. Natl. Acad. Sci. U.S.A., 106:3794-3799, 2009.
Alvarez-Erviti et al., Delivery of siRNA to the mouse brain by systemic injection of targeted exosomes. Nature Biotechnology, 29:341-345, 2011.
Austin-Ward & Villaseca, Gene therapy and its applications. Rev. Med. Chil., 126:838-845, 1998.
Baietti et al., Syndecan-syntenin-ALIX regulated the biogenesis of exosomes. Nat. Cell Biol., 14:677-685, 2012.
Bastos et al., Exosomes in cancer: Use them or target them? Semin. Cell Dev. Biol., 78:13-21, 2018.
Biankin et al., Pancreatic cancer genomes reveal aberrations in axon guidance pathway genes. Nature, 491:399-405, 2012.
Borges et al., TGF-beta1-containing exosomes from injured epithelial cells activate fibroblasts to initiate tissue regenerative responses and fibrosis. J. Amer. Soc. Nephrology, 24:385-392, 2013.
Bukowski et al., Signal transduction abnormalities in T lymphocytes from patients with advanced renal carcinoma: clinical relevance and effects of cytokine therapy. Clin. Cancer Res., 4:2337-2347, 1998.
Chang et al., Pancreatic cancer genomics. Current Opinion in Genetics & Development, 24:74-81, 2014.
Choi et al., The protein interaction network of extracellular vesicles derived from human colorectal cancer cells. J. Proteome Res., 11:1144-1151, 2012.
Christodoulides et al., Immunization with recombinant class 1 outer-membrane protein from Neisseria meningitidis: influence of liposomes and adjuvants on antibody avidity, recognition of native protein and the induction of a bactericidal immune response against meningococci. Microbiology, 144:3027-3037, 1998.
Clayton et al., Antigen-presenting cell exosomes are protected from complement-mediated lysis by expression of CD55 and CD59. Eur. J. Immunology, 33:522-531, 2003.
Collins et al., Oncogenic Kras is required for both the initiation and maintenance of pancreatic cancer in mice. J. Clinical Investigation, 122:639-653, 2012a.
Collins et al., Metastatic pancreatic cancer is dependent on oncogenic Kras in mice. PLoS One, 7:e49707, 2012b.
Colombo et al., Biogenesis, secretion, and intercellular interactions of exosomes and other extracellular vesicles. Annu. Rev. Cell. Dev. Biol., 30:255-289, 2014.
Combes et al., A new flow cytometry method of platelet-derived microvesicle quantitation in plasma, Thromb. Haemost., 77:220, 1997.
Cooper et al., Systemic exosomal siRNA delivery reduced alpha-synuclein aggregates in brains of transgenic mice. Movement Disorders, 29:1476-1485, 2014.
Croft et al., The Reactome pathway knowledgebase. Nuc. Acids Res., 42:D472-477, 2014. Davidson et al., Intralesional cytokine therapy in cancer: a pilot study of GM-CSF infusion in mesothelioma. J. Immunother., 21:389-398, 1998.
Du et al., A systematic analysis of the silencing effects of an active siRNA at all single-nucleotide mismatched target sites. Nuc. Acids Res., 33:1671-1677, 2005.
El-Andaloussi et al., Extracellular vesicles: biology and emerging therapeutic opportunities. Nature Reviews Drug Discovery, 12:347-357, 2013.
El-Andaloussi et al., Exosome-mediated delivery of siRNA in vitro and in vivo. Nature Protocols, 7:2112-2126, 2012.
Eser et al., Oncogenic KRAS signalling in pancreatic cancer. British Journal of Cancer, 111:817-822, 2014.
Gartel et al., A new method for determining the status of p53 in tumor cell lines of different origin. Oncology Research, 13:405-408, 2003.
Gomes-da-Silva et al., Lipid-based nanoparticles for siRNA delivery in cancer therapy: paradigms and challenges. Accounts of Chemical Research, 45:1163-1171, 2012.
Gonzales et al., Large-scale proteomics and phosphoproteomics of urinary exosomes. J. Amer. Soc. Nephrology, 20:363-379, 2009.
Gysin et al., Therapeutic strategies for targeting ras proteins. Genes & Cancer, 2:359-372, 2011.
Hanibuchi et al., Therapeutic efficacy of mouse-human chimeric anti-ganglioside GM2 monoclonal antibody against multiple organ micrometastases of human lung cancer in NK cell-depleted SCID mice. Int. J. Cancer, 78:480-485, 1998.
Harding et al., Endocytosis and intracellular processing of transferrin and colloidal gold-transferrin in rat reticulocytes: demonstration of a pathway for receptor shedding. European J. Cell Biol., 35:256-263, 1984.
Hazan-Halevy et al., Cell-specific uptake of mantle cell lymphoma-derived exosomes by malignant and non-malignant B-lymphocytes. Cancer Lett., 364:59-69, 2015.
Hellstrand et al., Histamine and cytokine therapy. Acta Oncol., 37:347-353, 1998.
Hingorani et al., Trp53R172H and KrasG12D cooperate to promote chromosomal instability and widely metastatic pancreatic ductal adenocarcinoma in mice. Cancer Cell, 7:469-483, 2005.
Hollander, Immunotherapy for B-cell lymphoma: current status and prospective advances. Front Immunol., 3:3, 2013.
Howlader et al., SEER Cancer Statistics Review, 1975-2011, National Cancer Institute. Bethesda, Md. On the World Wide Web at seercancergov/csr/1975_2011/, 2013.
Hruban et al., K-ras oncogene activation in adenocarcinoma of the human pancreas. A study of 82 carcinomas using a combination of mutant-enriched polymerase chain reaction analysis and allele-specific oligonucleotide hybridization. American J. Pathology, 143:545-554, 1993.
Huang et al., Epidermal growth factor receptor-containing exosomes induce tumor-specific regulatory T cells. Cancer Invest., 31:330-335, 2013.
Hui & Hashimoto, Pathways for Potentiation of Immunogenicity during Adjuvant-Assisted Immunizations with Plasmodium falciparum Major Merozoite Surface Protein 1. Infec. Immun., 66:5329-5336, 1998.
Ishihama et al., Exponentially modified protein abundance index (emPAI) for estimation of absolute protein amount in proteomics by the number of sequenced peptides per protein. Mol. Cell. Proteomics, 4:1265-1272, 2005.
Ji et al., Ras activity levels control the development of pancreatic diseases. Gastroenterology, 137:1072-1082, 82 el-6, 2009.
Johnsen et al., A comprehensive overview of exosomes as drug delivery vehicles—endogenous nanocarriers for targeted cancer therapy. Biochimica et Biophysica Acta, 1846:75-87, 2014.
Kahlert et al., Identification of Double Stranded Genomic DNA Spanning all Chromosomes with Mutated KRAS and p53 DNA in the Serum Exosomes of Patients with Pancreatic Cancer. J. Biol. Chem., 289:3869-3875, 2014.
Kaira et al., Comparative proteomics evaluation of plasma exosome isolation techniques and assessment of the stability of exosomes in normal human blood plasma. Proteomics, 13:3354-3364, 2013.
Kalluri, The biology and function of exosomes in cancer. J. Clin. Invest., 126:1208-1215, 2016.
Kamerkar et al., Exosomes facilitate therapeutic targeting of oncogenic KRAS in pancreatic cancer. Nature, 546:498-503, 2017.
Kowal et al., Biogenesis and secretion of exosomes. Current Opinion in Cell Biology, 29:116-125, 2014.
Kramer et al., The ribosome as a platform for co-translational processing, folding and targeting of newly synthesized proteins. Nat. Struct. Mol. Biol., 16:589-597, 2009.
Li et al., Exosomes derived from gefitinib-treated EGFR-mutant lung cancer cells alter cisplatin sensitivity via up-regulating autophagy. Oncotarget, 7:24585-24595, 2016.
Lim et al., EGFR Signaling Enhances Aerobic Glycolysis in Triple-Negative Breast Cancer Cells to Promote Tumor Growth and Immune Escape. Cancer Research, 76:1284-1296, 2016.
Liu et al., EGFR expression correlates with decreased disease-free survival in triple-negative breast cancer: a retrospective analysis based on a tissue microarray. Med. Oncol., 29:401-405, 2012.
Livak & Schmittgen, Analysis of relative gene expression data using real-time quantitative PCR and the 2(−Delta Delta C(T)) Method. Methods, 25:402-408, 2001.
Lorch et al., Role of DNA sequence in chromatin remodeling and the formation of nucleosome-free regions. Genes Dev., 28:2492-2497, 2014.
Luga et al., Exosomes mediate stromal mobilization of autocrine Wnt-PCP signaling in breast cancer cell migration. Cell, 151:1542-1556, 2012.
Ma et al., Structural basis for overhang-specific small interfering RNA recognition by the PAZ domain. Nature, 429:318-322, 2004.
Marcus & Leonard, FedExosomes: Engineering Therapeutic Biological Nanoparticles that Truly Deliver. Pharmaceuticals (Basel), 6:659-680, 2013.
Masuda et al., Role of epidermal growth factor receptor in breast cancer. Breast Cancer Res. Treat., 136:331-345, 2012.
Melo et al., Cancer exosomes perform cell-independent microRNA biogenesis and promote tumorigenesis. Cancer Cell, 26:707-721, 2014.
Melo et al., Glypican-1 identifies cancer exosomes and detects early pancreatic cancer. Nature, 523:177-182, 2015.
Merrick, Cap-dependent and cap-independent translation in eukaryotic systems. Gene, 332:1-11, 2004.
Morgillo et al., Mechanisms of resistance to EGFR-targeted drugs: lung cancer. ESMO Open, 1:e000060, 2016.
Morino et al., Eukaryotic translation initiation factor 4E (eIF4E) binding site and the middle one-third of eIF4GI constitute the core domain for cap-dependent translation, and the C-terminal one-third functions as a modulatory region. Mol. Cell Biol., 20:468-477, 2000.
Nagai et al., Chromatin potentiates transcription. Proc. Natl. Acad. Sci. U.S.A., 114:1536-1541, 2017.
Nakai et al., A perspective on anti-EGFR therapies targeting triple-negative breast cancer. Am. J. Cancer Res., 6:1609-1623, 2016.
Normanno et al., Epidermal growth factor receptor (EGFR) signaling in cancer. Gene, 366:2-16, 2006.
Ozdemir et al., Depletion of carcinoma-associated fibroblasts and fibrosis induces immunosuppression and accelerates pancreas cancer with reduced survival. Cancer Cell, 25:719-734, 2014.
Pardoll, Cancer immunotherapy through checkpoint blockade: the future of cancer treatment. Medicographia, 36:274-284, 2014.
Pathan et al., FunRich: An open access standalone functional enrichment and interaction network analysis tool. Proteomics, 15:2597-2601, 2015.
Pecot et al., Therapeutic Silencing of KRAS using Systemically Delivered siRNAs. Molecular Cancer Therapeutics, 13:2876-2885, 2014.
Peinado et al., Melanoma exosomes educate bone marrow progenitor cells toward a pro-metastatic phenotype through MET. Nature Medicine, 18:883-891, 2012.
Perdigao et al., Unexpected features of the dark proteome. Proc. Natl. Acad. Sci. U.S.A., 112:15898-5903, 2015.
Pico de Coana et al., Checkpoint blockade for cancer therapy: revitalizing a suppressed immune system. Trends in Molecular Medicine, 21:482-492, 2015.
Pisitkun et al., Identification and proteomic profiling of exosomes in human urine. Proc. Natl. Acad. Sci. U.S.A., 101:13368-13373, 2004.
Poliseno et al., A coding-independent function of gene and pseudogene mRNAs regulates tumour biology. Nature, 465:1033-1038, 2010.
Poulin et al., 4E-BP3, a new member of the eukaryotic initiation factor 4E-binding protein family. J. Biol. Chem., 273:14002-14007, 1998.
Qin et al., Interferon-beta gene therapy inhibits tumor formation and causes regression of established tumors in immune-deficient mice. Proc. Natl. Acad. Sci. U.S.A., 95:14411-14416, 1998.
Rachagani et al., Activated KrasG12D is associated with invasion and metastasis of pancreatic cancer cells through inhibition of E-cadherin. Br. J. Cancer, 104:1038-1048, 2011.
Rao & Cruz, Effects of confinement on the structure and dynamics of an intrinsically disordered peptide: a molecular-dynamics study. J. Phys. Chem. B., 117:3707-3719, 2013.
Raposo & Stoorvogel, Extracellular vesicles: exosomes, microvesicles, and friends. J. Cell Biol., 200:373-383, 2013.
Rejiba et al., K-ras oncogene silencing strategy reduces tumor growth and enhances gemcitabine chemotherapy efficacy for pancreatic cancer treatment. Cancer Science, 98:1128-1136, 2007.
Ronquist et al., Prostasomes from four different species are able to produce extracellular adenosine triphosphate (ATP). Biochim Biophys. Aca, 1830:4604-4610, 2013a.
Ronquist et al., Human prostasomes express glycolytic enzymes with capacity for ATP production. Am. J. Physiol. Endocrinol. Metab., 304:E576-582, 2013b.
Rothstein et al., Targeting signal 1 through CD45RB synergizes with CD40 ligand blockade and promotes long term engraftment and tolerance in stringent transplant models. J. Immunol., 166:322-329, 2001.
Seshacharyulu et al., Targeting the EGFR signaling pathway in cancer therapy. Expert Opin. Ther. Targets, 16:15-31, 2012.
Siegel et al., Cancer statistics, 2014. CA: A cancer journal for clinicians, 64:9-29, 2014.
Simoes et al., Cationic liposomes for gene delivery. Expert Opinion on Drug Delivery, 2:237-254, 2005.
Sinkovics, The cell survival pathways of the primordial RNA-DNA complex remain conserved in the extant genomes and may function as proto-oncogenes. Eur. J. Microbiol. Immunol. (Bp), 5:25-43, 2015.
Skogberg et al., Characterization of human thymic exosomes. PLoS ONE, 8:e67554, 2013.
Smakman et al., Dual effect of Kras(D12) knockdown on tumorigenesis: increased immune-mediated tumor clearance and abrogation of tumor malignancy. Oncogene, 24:8338-8342, 2005.
Smith et al., Local protein synthesis in neurons. Curr. Biol., 11:R901-903, 2001.
Song et al., Cancer Cell-derived Exosomes Induce Mitogen-activated Protein Kinase-dependent Monocyte Survival by Transport of Functional Receptor Tyrosine Kinases. J. Biol. Chem., 291:8453-8464, 2016.
Steward & Levy, Preferential localization of polyribosomes under the base of dendritic spines in granule cells of the dentate gyrus. J. Neurosci., 2:284-291, 1982.
Sun et al., Characterization of the mutations of the K-ras, p53, p16, and SMAD4 genes in 15 human pancreatic cancer cell lines. Oncology Reports, 8:89-92, 2001.
Thery et al., Exosomes: composition, biogenesis and function. Nature Reviews Immunology, 2:569-579, 2002.
Thery et al., Isolation and characterization of exosomes from cell culture supernatants and biological fluids. Current Protocols in Cell Biology, Chapter 3, Unit 3 22, 2006.
Tomas et al., EGF receptor trafficking: consequences for signaling and cancer. Trends Cell Biol., 24:26-34, 2014.
Ung et al., Exosome proteomics reveals transcriptional regulator proteins with potential to mediate downstream pathways. Cancer Sci., 105:1384-1392, 2014.
Valadi et al., Exosome-mediated transfer of mRNAs and microRNAs is a novel mechanism of genetic exchange between cells. Nature Cell Biology. 9:654-659, 2007.
van den Boom et al., Exosomes as nucleic acid nanocarriers. Advanced Drug Delivery Reviews, 65:331-335, 2013.
van der Meel et al., Extracellular vesicles as drug delivery systems: Lessons from the liposome field. J. Controlled Release, 195:72-85, 2014.
Wahlgren et al., Plasma exosomes can deliver exogenous short interfering RNA to monocytes and lymphocytes. Nucleic Acids Research, 40:e130, 2012.
Westphal et al., EGFR as a Target for Glioblastoma Treatment: An Unfulfilled Promise. CNS Drugs, 31:723-735, 2017.
Weyrich et al., Change in protein phenotype without a nucleus: translational control in platelets. Semin. Thromb. Hemost., 30:491-498, 2004.
Willms et al., Cells release subpopulations of exosomes with distinct molecular and biological properties. Sci. Rep., 6:22519, 2016.
Wykes & Lewin, Immune checkpoint blockade in infectious diseases. Nat. Rev. Immunology, 18:91-104, 2018.
Xue et al., Small RNA combination therapy for lung cancer. Proc. Natl. Acad. Sci. U.S.A., 111:E3553-3561, 2014.
Yamada et al., Cell Infectivity in Relation to Bovine Leukemia Virus gp51 and p24 in Bovine Milk Exosomes. PLoS ONE, 8:e77359, 2013.
Ying et al., Oncogenic Kras maintains pancreatic tumors through regulation of anabolic glucose metabolism. Cell, 149:656-670, 2012.
Yuan et al., Development of siRNA payloads to target KRAS-mutant cancer. Cancer Discovery, 4:1182-1197, 2014.
Zhang et al., Exosome-delivered EGFR regulates liver microenvironment to promote gastric cancer liver metastasis. Nat. Commun., 8:15016, 2017.
Zhang et al., A mechanism for the upregulation of EGF receptor levels in glioblastomas. Cell. Rep., 3:2008-2020, 2013.
Zilfou & Lowe, (2009). Tumor suppressive functions of p53. Cold Spring Harb. Perspect. Biol., 1:a001883, 2009.
Zorde Khvalevsky et al., Mutant KRAS is a druggable target for pancreatic cancer. Proc. Natl. Acad. Sci. U.S.A., 110:20723-20728, 2013.

What is claimed is:

1. A method of treating a disease or disorder in a patient in need thereof, the method comprising:
(a) obtaining exosomes having a growth factor receptor on their surface;
(b) transfecting the exosomes with a nucleic acid encoding a therapeutic protein;
(c) administering the transfected exosomes to a patient; and
(d) providing a growth factor gradient at a site of the disease or disorder to attract the exosomes to the site and stimulate production of the therapeutic protein at the site, thereby treating the disease in the patient.

2.-21. (canceled)

22. A method of treating a disease or disorder in a patient in need thereof, the method comprising:
(a) obtaining exosomes having a growth factor receptor on their surface;
(b) transfecting the exosomes with a therapeutic agent;
(c) administering the transfected exosomes to a patient; and
(d) providing a growth factor gradient at a site of the disease or disorder to attract the exosomes to the site and deliver the therapeutic agent to the site, thereby treating the disease in the patient.

23. (canceled)

24. The method of claim 22, wherein the exosomes obtained in step (a) are obtained from a body fluid sample obtained from the patient.

25. The method of claim 24, wherein the body fluid sample is blood, lymph, saliva, urine, cerebrospinal fluid, bone marrow aspirates, eye exudate/tears, or serum.

26. The method of claim 22, wherein the therapeutic agent is a therapeutic protein, an antibody, an inhibitory RNA, a gene editing system, or a small molecule drug.

27. The method of claim 26, wherein the therapeutic agent is an antibody, wherein the antibody binds an intracellular antigen.

28. (canceled)

29. The method of claim 26, wherein the therapeutic agent is an inhibitory RNA, wherein the inhibitory RNA is a siRNA, shRNA, miRNA, or pre-miRNA.

30. The method of claim 26, wherein the therapeutic agent is a gene editing system, wherein the gene editing system is a CRISPR/Cas system.

31. The method of claim 26, wherein the therapeutic agent is a therapeutic protein, wherein the therapeutic protein is a kinase, a phosphatase, or a transcription factor.

32. The method of claim 26, wherein the therapeutic agent is a therapeutic protein, wherein the therapeutic protein corresponds to a wildtype version of a protein that is mutated or inactivated in a cell at the site of the disease or disorder.

33. The method of claim 26, wherein the therapeutic agent is a therapeutic protein, wherein the therapeutic protein corresponds to a dominant negative version of a protein that is hyperactive in a cell at the site of the disease or disorder.

34. (canceled)

35. The method of claim 22, wherein the disease or disorder is cancer, an injury, an autoimmune disorder, a neurological disorder, a gastrointestinal disorder, an infectious disease, a kidney disease, a cardiovascular disorder, an ophthalmic disorder, a skin disease or disorder, a urogenital disorder, or a bone disease or disorder.

36. The method of claim 35, wherein the disease or disorder is cancer, wherein the cancer is a breast cancer, lung cancer, head & neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer or skin cancer.

37. The method of claim 36, wherein the site of the disease or disorder is a tumor.

38. The method of claim 36, wherein the cancer is metastatic, wherein the site of the disease or disorder is a metastatic node.

39. (canceled)

40. The method of claim 22, wherein the disease or disorder is cancer, wherein the therapeutic agent is a therapeutic protein, and wherein the therapeutic protein is a tumor suppressor.

41. The method of claim 22, wherein the disease or disorder is cancer, wherein the therapeutic agent is an inhibitory RNA targeting an oncogene.

42. The method of claim 22, wherein the exosomes comprise CD47 on their surface.

43. (canceled)

44. The method of claim 22, further comprising administering at least a second therapy to the patient, wherein the second therapy comprises a surgical therapy, chemotherapy, radiation therapy, cryotherapy, hormonal therapy, or immunotherapy.

45. (canceled)

46. The method of claim 22, wherein the exosomes are comprised in tissue scaffold matrix.
